(19) United States
(12) Patent Application Publication
(10) Pub. No.: US 2006/0252707 A1
(43) Pub. Date: Nov. 9, 2006

(54) Pharmaceutical composition for the treatment and/or the prevention of atherosclerosis from infectious origin

(75) Inventors: Carol Deby, Liege (BE); Ginette Dupont, Liege (BE); Didier Serteyn, Tavier (BE)

Correspondence Address: FITCH EVEN TABIN AND FLANNERY, 120 SOUTH LA SALLE STREET, SUITE 1600, CHICAGO, IL, 60603-3406, US

(73) Assignee: UNIVERSITE DE LIEGE, Liege (BE)

(21) Appl. No.: 10/558,503

(22) PCT Filed: May 28, 2004
(86) PCT No.: PCT/BE04/00078

§ 371 Date: Nov. 28, 2005

(30) Foreign Application Priority Data
May 28, 2003 (EP) 03447132.6

(51) Int. Cl. A61K 31/7048; A61K 31/573; A61K 31/353; A01N 31/08

(52) U.S. Cl. 514/27; 514/171; 514/733; 514/456

(57) ABSTRACT

The present invention is related to a pharmaceutical composition suitable for the treatment and/or the prevention of atherosclerosis from infectious origin, which comprises an adequate pharmaceutical carrier, a corticosteroid and a stilbene-type alexin, preferably further comprising a flavonoid to regenerate the stilbene and/or to increase the effect of the latter. The latter compositions are highly suitable for long-term therapies like the treatment of atherosclerosis from infectious origin.

### FIELD OF THE INVENTION

The present invention is related to a pharmaceutical composition for the treatment and/or the prevention of atherosclerosis from infectious origin, especially atherosclerosis induced by intracellular micro-organisms, in particular Chlamydia pneumoniae.

### BACKGROUND OF THE INVENTION

Atherosclerosis is responsible for coronary diseases, myocardial infarction, cerebral sclerosis and stroke (stroke is the current designation for apoplexy or cerebral congestion).

There is a total of 17 millions deaths per year, related to atherosclerosis; atherosclerosis is the first cause of death in Europe, and nothing lets believe that the mortality rate due to atherosclerosis will decrease, at least not in our western countries. Many forms of atherosclerosis could be related to infections by intracellular microorganisms in subjects with a possible hereditary insufficient immunological defense against these microorganisms: until now no satisfactory vaccines have been obtained.

Since 1981, it is recognized that atherosclerosis is an inflammatory disease (Ross, 1999, Engl. J. Med. 340: 115-126) and not a degenerative process as claimed in the past. It can thus be treated and cured or stopped.

The atherosclerotic process is a long term process. It is mainly a lipid infiltration process after a proteolytic and oxidative aggression of the arterial walls. Protein and lipid cellular debris form a gruel, rich in cholesterol and cholesterol esters, starting an atherosclerotic plaque. These areas are then invaded by conjunctive fibres and non oriented muscular fibres.

In the gruel stage, the atherosclerotic plaque is fragile and tends to break (plaque rupture followed by thrombosis). When invaded by fibres, the plaque becomes more stable but diminishes the vascular lumen, limiting the blood flow (Ross, 1999, see above). These alterations are the consequence of a pathological activation of blood white cells, particularly the monocytes which cross the vascular wall by diapedesis. Monocytes are rich in lysosomes and NADPH oxidase. Lysosomes contain proteolytic enzymes (metalloproteinases), and NADPH oxidase produces the superoxide anion, precursor of most of the activated oxygen species that are responsible for the oxidative attacks (Babior, 1999, Blood 93: 1464-76).

The cellular process can be described as follows: for a reason that has remained not understood for a long time, the blood monocyte crosses the endothelium and enters the intima of the arterial wall. In the intima, the monocyte morphology changes: the cell loses its spherical form, considerably increases in size and becomes an amiboid cell: the monocyte has been transformed into a macrophage. In the neighbouring tissues, the macrophage releases reactive oxygen species such as hydrogen peroxide (which transforms into more reactive species), secretes metalloproteinases and ingests by phagocytosis blood lipoproteins which have reached the intima by crossing the endothelium.

The metalloproteinases form the proteic part of the gruel; the ingested lipoproteins are oxidized and form the lipid part of the gruel. Macrophages accumulate the main part of lipoprotein cholesterol and cholesterol esters and become the foam cell, a great cell packed with lipid granules, characteristic of the atherosclerotic lesion.

An important fact is the release of messenger substances by the macrophages. These messengers include cytokines, of which the main role is to stimulate from a distance other blood monocytes, leading them to cross the arterial wall at the level of the growing atherosclerotic focus (chimiotaxis). The cytokines, mainly TNFα, IL-1β and IL-8, attract not only circulating monocytes, but also circulating polymorphonuclear neutrophils. Neutrophils possess an important enzyme, myeloperoxidase, which transforms hydrogen peroxide into chlorinated derivatives, mainly hypochlorous acid, chloramines, and even chlorine, which are highly destructive for tissues.

During many years, the reason of attraction and excitation of white cells in an arterial site, responsible for its alteration into a growing atherosclerotic plaque was unknown or attributed to mysterious mechanisms oxidizing the circulating lipids, mainly cholesterol. Now, more and more evidence is found for the fact that the presence of an endocellular parasitic bacterium, infecting even few arterial wall cells (endothelial and smooth muscle cells, fibroblasts), is sufficient for the excitation of the circulating monocytes by bacterial toxins. The parasitic bacterium is Chlamydia pneumoniae. Other intracellular micro-organisms such as Mycoplasma are suspected to play the same role.

Atherosclerosis is thus now considered as a disease of infectious origin. In 1988, Saikku et al. (Lancet 2: 983-986) hypothesised that the infectious agent was the intracellular bacterium Chlamydia pneumoniae. After a lot of understandable reluctance, the number of scientists that agree with Saikku's hypothesis started growing. A consensus recognising the infectious origin to many atherosclerosis cases started from 1998, and most of researchers now agree with this infectious origin of atherosclerosis.

Chlamydiae are entirely intracellular or endocellular micro-organisms, which are totally dependent from the host.

Four types of Chlamydiae are known:
Chlamydia trachomatis: responsible for an eye-disease which is transmitted by flies, and also responsible for sterility, sexually transmitted,
Chlamydia (Chlamydophila) pneumoniae: transmitted through aerosol (cough),
Chlamydia psittaci: affects birds (psittacosis), can be transmitted to humans (ornithosis),
Chlamydia pecorum (bovine): responsible for respiratory tract disease, can be transmitted to humans.

Chlamydiae exist as elementary bodies (EB, latent form) and reticular bodies (RB, active form). It is the active form of the bacterium (the RB) that emits toxins and that is responsible for auto-destruction of the host. Propagation of the infection by Chlamydiae is due to the elementary bodies, which are phagocytized by macrophages. But macrophages are unable to <<digest>> the micro-organism which, moreover, possesses the capacity to delay macrophage apoptosis. Other cells can be infected by Chlamydiae (e.g. endothelial cells and smooth muscle cells).

Chlamydiae are highly sensitive to a particular type of antibiotics, the macrolides. But monocytes are real <<sanctuaries>> where the elementary bodies are sheltered from antibiotics. It means that macrolides are potent weapons for slowering evoluting atherosclerotic processes (able to provoke arterial thrombosis). But, to be able to eradicate this intracellular parasite, therapy by antibiotics has to be continued for years and years with arrests becoming shorter and shorter.

To avoid the use of antibiotics, several research teams are trying to develop vaccines. Until now, these research efforts have been unsuccessful because Chlamydiae are not very antigenic.

As an alternative to a therapy with antibotics, one could consider slowing down the development of vascular wall alterations with anti-inflammatory drugs. Corticosteroids are the more active anti-inflammatory drugs. In literature, however, there exists controversy about the effects of corticosteroids on the development of atherosclerosis. These discrepancies are explained by showing that corticosteroids can enhance the production of reactive oxygen species by the monocytes that are excited by Chlamydia toxins. Therefore, the beneficial anti-inflammatory and anti-atherosclerosis effects of these steroids are cancelled.

The main anti-inflammatory effects of corticosteroids are:
1. the inhibition of cytokine production; steroids block so the exponential amplification of monocyte auto-stimulation.
2. the suppression of the adhesion molecules produced by the excited cells and responsible for the adhesion to the vascular wall.

Below, the mechanism of Chlamydia-induced atherosclerosis, in its cerebral form as well as in the other forms (in coronary arteries or in other organs, such as kidney) is described: defence cells (monocytes) are continuously and excessively stimulated by bacterial toxins, become macrophages, and penetrate the arterial walls, which are progressively transformed into fatty streaks, developing then into an atherosclerotic plaque. An oxidizing enzyme (NADPH-oxidase) and metalloproteinases of the monocytes/macrophages are mainly responsible for the development of this process. Thus, bacteria appear to be initiating agents of arterial destruction, few bacterial toxins being sufficient to excite monocytes. Then, a loop of amplification starts between the white cells, which excite each other by uncontrolled secretion of cytokines.

To obtain an efficient treatment of atherosclerosis, it is thus not only necessary to eradicate (i.e. by administration of antibiotics) the causative micro-organisms. It is also imperial to reduce the exciting effect of the infecting micro-organisms on monocyte cells that have been stimulated by said micro-organisms.

### STATE OF THE ART

Antibiotics, and more in particular macrolides, have been used with success to control and/or eradicate Chlamydiae, causative agents of atherosclerosis from infectious origin, but only when applied more or less continuously over a period of several years, with considerable collateral effects such as intestinal troubles and fatigue as consequence.

Corticosteroids and in particular glucocorticoids are known as potent anti-inflammatory drugs and have been proposed in the treatment of inflammatory diseases such as atherosclerosis. There are, however, a few drawbacks that hamper their straightforward application in the treatment of atherosclerosis from infectious origin. It has been described for instance that corticosteroids can enhance the production of reactive oxygen species by the monocytes excited by Chlamydia toxins. A prolonged corticotherapy would even result in a higher atherosclerosis incidence (Kalbak, 1972, Ann Rheum Dis. 31: 196-200; Troxler et al., 1977, Atherosclerosis 26: 151-162). Certainly in the case of elevated corticosteroid doses, a prolonged corticotherapy can induce accumulation of abdominal fat, insulin-resistance, arterial hypertension, hyperlipidemia (Nashel, 1986, Am J Med 80: 925-929; Despres et al., 1990, Arteriosclerosis 10: 497-511).

It is also known that stilbenes have pharmaceutical applications. The stilbene-type phyto-alexin resveratrol is used for a long time in oriental traditional medicament to treat inflammatory phenomena. Resveratrol as well as its analogues (hydroxylated and methoxylated analogues of resveratrol) are now considered as useful to slow down or inhibit the development of several forms of cancers, such as colorectal cancer. Resveratrol is further known as anti-oxidant.

U.S. Pat. No. 6,048,903 proposes the use of trans-resveratrol to reduce the level of light density lipoproteins (LDL) and thereby the risk of hypercholesterolemia. High cholesterol levels are a risk factor for atherosclerosis but are no causative agent thereof.

U.S. Pat. No. 6,211,247 discloses methods of preventing restenosis (an accelerated form of atherosclerosis of non-infectious origin) and the recurrence or progression of coronary heart disease based on the addition of cis-resveratrol and/or trans-resveratrol.

WO 02/32410 discloses methods for treating inflammatory respiratory disorders with resveratrol, possibly in combination with corticosteroids or glucococorticoids.

Oestrogens like the synthetic oestrogen diethylstilbesterol (DES) and glucocorticoids like prednisolone and dexamethasone have been proposed for the treatment of established atherosclerosis and the prevention of atherosclerosis of cholesterol-fed rabbits, and more in particular edematous arterial reactions in cholesterol-fed rabbits, rhesus monkeys, dogs, guinea pigs and rats (Shimamoto, 1968, Acta Pathologica Japonica 19: 15-43). Edematous arterial reactions are considered as initial stages of atherosclerosis. Numano (1980, Japanese Circulation Journal 44: 55-68) proposed the above compounds for the treatment or correction of hyperlipidemia. The treatment and/or prevention of human atherosclerosis from infectious origin are not discussed in these documents.

DES and Prednisolone (Pr), administered alone or in combination, would be able to reduce the enzyme efflux from skeletal muscle and would therefore be effective in the treatment of for instance Duchenne's muscular dystrophy (Morgan et al., 1976, Clinical Research 24: page 520 A; Cohen et al., 1977, Journal of Medicine 8: 123-134).

DES, though structurally similar to resveratrol has completely different biological activities. DES is known to be carcinogenic, has an oxidising effect on fats (Gued et al., 2003, Oncol Rep 10: 739-743), is a strong oestrogen that induces chemical castration in males, impotency, mammary hypertrophy etc (Clemens, 1974, Adv Behav Biol 11: 23-53), is known to cause foetal anomalies etc. In other words, it possesses many properties that make it unsuitable for use in the prevention and/or treatment of atherosclerosis from infectious origin and/or in other long-term therapeutic regimens.

Long-term therapy with cortisone or corticosteroids is known to affect the natural resistance of humans to Chlamyida infections and/or to increase C. pneumoniae growth in vitro (Grayston, 1998, Circulation 97: 1669-1770; Malinverni et al., 1995, J. Infect. Dis. 172: 593-594; Laitinen et al., 1996, Infect. Immun. 64: 1488-1490; Tsumura et al., 1996, J. Clin. Microbiol. 34: 2379-2381).

There is a lack of effective therapeutic regimens for the treatment and/or prevention of atherosclerosis from infectious origin, in particular human atherosclerosis induced by a Chlamydia infection. There is no indication in the prior art on how to reduce and/or prevent among others the exciting effect of the infecting micro-organisms on monocyte cells that have been stimulated by said micro-organisms.

### AIMS OF THE INVENTION

The present invention aims to provide a new (pharmaceutical) composition for improving the treatment and/or the prevention of atherosclerosis from infectious origin, especially atherosclerosis induced by intracellular pathogenic micro-organisms, in particular the bacteria Chlamydia pneumoniae.

A preferred aim of the present invention is to provide such pharmaceutical composition, which effectively treats and/or prevents said disease by reducing or suppressing the exciting effect (leading to modification and destruction of arterial intima and plaque formation) of a toxin induced by said intracellular micro-organism, especially bacteria Chlamydia pneumoniae.

A further aim of the present invention is to propose such pharmaceutical composition that reduces monocyte activity in a mammal patient and therefore, reduces the effect of the inflammatory disease.

A further aim is to propose such composition, which comprises a very low dosage of two associated compounds and therefore reduce possible side effects of said one of the components of the pharmaceutical composition in a mammal patient.

Still a further aim of the present invention is to propose a pharmaceutical formulation that is adequate for a non-invasive administration, such as transcutaneous administration.

A last aim of the invention is to propose suitable and effective therapeutic regimens with the least possible side-effects.

### SUMMARY OF THE INVENTION

A first aspect of the present invention is related to a new pharmaceutical composition for the treatment and/or the prevention of atherosclerosis from infectious origin, which comprises an adequate pharmaceutical carrier, a corticosteroid, more preferably a glucocorticoid, and a stilbene-type phytoalexin (such as resveratrol (cis or trans form), (or a pharmaceutical acceptable salt or its metabolite) and one or more polyphenol(s).

The pharmaceutical composition may also comprise an ester, an amide, a mono- or disaccharide conjugate of resveratrol. Examples and structures of such compounds are given in WO 02/32410 (incorporated by reference herein). Preferably, the metabolite of resveratrol is piceatanol.

In the composition according to the invention, preferred corticosteroids are methylprednisolone, hydrocortisone or derivatives thereof. Methylprednisolone (MPr) is preferred over hydrocortisone (HCT) because it does not increase the oxidant activity of macrophages and thus the peroxidation of lipids at all.

Preferably, the composition comprises prednisolone or hydrocortisone in a concentration range of about 10⁻⁶ to about 10⁻⁷ M, and the stilbene-type phyto-alexin in a concentration range of about 10⁻⁵ M to about 10⁻⁶ M.

It was surprisingly found that the addition of polyphenols especially flavonoids such as flavan-3-ol (formula 1) and isoflavan-3-ol derivatives (such as for example catechin, epicatechin, gallocatechin, leucocyanidin) and flavanone (formula 2) derivatives (such as for example, rutin, quercetin, hesperidin, kaempferin, myricetin, apigenin, diosmin, luteolin, fisetin, troxerutin) remarkably improved the effectiveness of the above compositions. Useful flavonoids are those of formula (1) or formula (2):

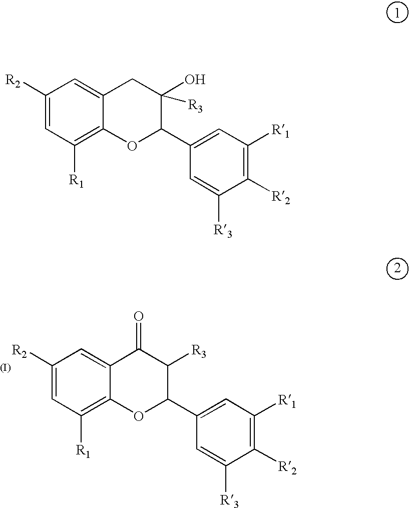

herein R₁ can be H, OH; R₂ can be H, OH, O-sugar residues (preferably said O-sugar residues are pentoses or hexoses); R₃ can be H, OH; R′₁ can be H, OH, OCH₃; R′₂ can be H, OH, OCH₃, OCH₂CH₂OH; and R′₃ can be H, OH, OCH₂CH₂OH.

Rutin may be preferred for pharmaceutical preparations because it is long known to be non-toxic, especially at the concentration ranges proposed.

Resveratrol, polyphenols and flavonoids are known to be present in grapes, wine, especially in red wine, and in many plants (such as polygonum cuspidatum) which may serve as source for these components.

Surprisingly, the inventors discovered that the flavonoid can prevent and/or reduce to a great extent the otherwise rapid degradation (even when stored in the dark, or kept at low temperatures etc.) of stilbene-type phyto-alexins such as resveratrol. Rutin and/or quercetin have very little effect on its own apart from the ability to regenerate compounds such as resveratrol. Advantageously, concentrations as low as 10⁻⁶ M were found sufficient to achieve this effect. The adding of a flavonoid increases the actual shelf-life of the composition(s), which is particularly advantageous in the case of pharmaceutical preparations.

It was further found that a composition that comprises a flavonoid as further active ingredient—such as rutin and/or quercetin—is more effective than a composition comprising as ingredients a corticosteroid such as hydrocortisone or methylprednisolone and a stilbene-type phyto-alexin such as resveratrol, piceatanol or their salts, even when freshly prepared. Rutin and/or quercetin, and other flavonoids, are thus not only able to regenerate compounds like resveratrol, but also seem to re-enforce or increase the effect of the latter. There is thus a synergistic effect between these compounds :Resveratrol (and piceatanol) seems to act by inhibition of the protein kinase C (PKC), which triggers the activity of the enzyme responsible for superoxide anion production, the NADPH-oxidase. This activity of resveratrol (and piceatanol) is linked to its capacity to reduce some oxidant functions on the system PKC-NADPH-oxidase. By acting so, resveratrol becomes oxidized and is consumed. Flavonoids intervene here by reducing oxidized resveratrol, regenerating so active resveratrol. This regenerating activity of flavonoids on resveratrol is the result of the respective redox potentials of flavonoids and resveratrol. Flavonoids can reduce oxidized resveratrol but cannot act directly on the PKC-NADPH-oxidase system. This type of redox equilibrium is frequent in biology. The flavonoids act by the same regenerating mechanism to protect resveratrol during shelf-live, and more than one flavonoid molecule can be added to the composition.

Thanks to the association of rutin and/or quercetin and/or polyphenols (preferably flavonoids) to resveratrol and/or piceatanol, the concentration of the latter can be kept below a level whereby they would exert an estrogenic effect (Gehm et al.,1997, Proc Natl Acad Sci USA 94: 14138-43; Basly et al., 2000, Life Sci 66: 769-77; Bowers et al., 2000, Endocrinology 141: 3657-63; Bhat et al., 2001, Cancer Res 61: 7456-63). It is preferred to use the weakest dosages of resveratrol or resveratrol combined with the polyphenols (preferably the flavonolds above mentioned) possible because the resveratrol therapy is normally scheduled for longer periods, up to several years.

Such compositions are particularly suitable for the treatment and/or prevention of atherosclerosis from infectious origin, preferably human atherosclerosis from infectious origin. Atherosclerosis may hereby be induced by endocellular micro-organisms such as Chlamydiae, Mycoplasmae, Bartonellae and/or CMV.

Below, some guidelines are given with respect to preferred concentrations and/or preferred concentration ranges for each of the active components.

In the above compositions, the corticosteroid, which preferably is methylprednisolone, is used at low concentrations. With <<low concentration>> is meant a concentration low enough to avoid side-stimulating effects of this drug on reactive oxygen production by monocytes, and low enough to avoid reactivation of the endocellular micro-organisms—especially Chlamydiae—that are at the basis of atherosclerotic events. Suitable corticosteroid concentrations, more in particular suitable methylprednisolone concentrations lie in the range of about 10⁻⁶ M to about 10⁻⁵ M, with 10⁻⁷ M being preferred.

Also the stilbene-type phytoalexins—e.g. resveratrol, piceatanol or their respective salts—are preferably used in a low concentration (about 10⁻⁵ M to about 10⁻⁶ M) to avoid and/or to reduce estrogenic effects. The most preferred concentration is 10⁻⁶ M.

Rutin and/or quercetin are preferably used in a concentration range of about 10⁻⁵ M to about 10⁻⁶ M. The most preferred concentration is

The composition that was found most effective comprises methylprednisolone in a concentration of about 10⁻⁶ M to about 10⁻⁷ M, resveratrol in a concentration of about 10⁻⁵ M to about 10⁻⁶ M and rutin (or other polyphenols or flavonoids) in a concentration of about 10⁻⁵ M to about 10⁻⁶ M.

In the pharmaceutical composition according to the invention, the association of the active ingredients or compounds could be used to obtain unexpectedly the reduction of the monocytes/macrophages activity, implicating the development of atherosclerosis from infectious origin.

According to the invention, the micro-organisms implicated in the development of atherosclerosis from infectious origin is a bacterium, a virus, a mycoplasma or an intracellular parasite such as Chlamydiae, Mycoplasmae, Bartonellae and/or CMV.

In particular, the atherosclerosis from infectious origin treated or prevented by the pharmaceutical composition according to the invention is an atherosclerosis induced by the bacteria Chlamydia pneumoniae.

The inventors have discovered that the stilbene-type phyto-alexin resveratrol inhibits the activation of NADPH-oxidase, activation that follows the assembly of constitutive subunits of the NADPH-oxidase enzyme. This assembly is the first step in the pathway leading to the production of noxious oxidant species within the monocytes. Furthermore, these oxidant species are part of the roots of the primary atherosclerotic lesion: the <<foam cell>> (a cell full of lipids originating from lipoproteins that have been engulfed by monocytes and that have been oxidized by the oxidant species produced by these monocytes).

Therefore, the invention is based upon the synergic effects through a combination of (1) a stilbene-type phyto-alexin, and more preferably resveratrol, which strongly slows the generation of oxidant species, with (2) a corticosteroid such as prednisolone (or a derivative thereof), which, by the presence of said stilbene, only exerts favourable effects, mainly the reduction of adhesion molecules and of cytokine production. The use of said stilbene ensures a significant lowering of the corticosteroid therapeutic doses (up to 10⁻⁷ M), which become too weak to cause undesirable collateral effects such as immunosuppression phenomenons, oedemas, diabetes, etc.

These synergistic effects are even more pronounced when a polyphenol (flavonoid) is added in addition to the corticosteroid and a stilbene-type phyto-alexin. The polyphneol, preferably flavonoid prevents degeneration and loss of activity of the phyto-alexin and re-enforces or increases its effect by redox regeneration of the phyto-alexin.

Based on a discovery of Kalayoglu et al. (1998, J. Infect. Dis. 177: 725-729), the following in vitro cellular model is proposed for the analysis of atherosclerosis: the transformation of human monocytes into macrophages in the presence of Chlamydia.

On this model, the inventors have studied mainly the synthesis of reactive oxygen species by these excited monocytes. In a first step, they demonstrated that:
the intracellular micro-organism (Chlamydia) stimulates the production of reactive oxygen species by THP-1 cells (a human monocyte cell line), increasing twice or three times this production.
the enzyme that is responsible for this reactive oxygen production, is NADPH-oxidase.

Contrary to what Kalayoglu et al. believed, the inventors have established that the monocytes produce oxidant species by the classical way of NADPH-oxidase, an enzyme which permits the mono-electronic reduction of oxygen, forming superoxide anion, which, in turn, forms either hydrogen peroxide (spontaneously), or peroxynitrite y reaction with nitric oxide produced by the NO synthase.

From hydrogen peroxide (H₂O₂), various very reactive radical species are derived. From peroxynitrite (ONOO⁻), a lot of other radical molecules are produced.

The pharmaceutical compositions according to the invention may be presented in a specific formulation and should comprise an adequate pharmaceutical carrier for administration to a mammal patient, including a human patient. Preferably said administration is obtained by the transcutaneous route (preferably an administration by a patch).

The use of a patch is proposed because a stilbene-type phyto-alexin such as resveratrol is an unstable and photosensitive drug. The administration by patch will protect the active compounds.

The glucocorticoid administration at very low doses (<0,5 mg/day) could be intermittent, owing to the use of 2 kinds of patches: a patch of a stilbene (resveratrol) alone administered daily, and a patch of a stilbene (resveratrol) associated with a corticosteroid (methylprednisolone), administered intermittently.

Preferably, the patches also include one or two polyphenols (flavonoids), which are capable of regenerating and/or increasing the effect of the stilbene.

A further aspect of the invention therefore concerns suitable preparations and/or formulations based on the above pharmaceutical compositions, for instance patches with one or more of the active ingredients.

The above-proposed formulations and/or preparations may be provided in the form of a kit or a package containing one or more unit dosages. The kit may be a kit-of-parts, and may further comprise suitable dosages of an antibiotic, more in particular a suitable macrolide. A suitable macrolide is one that is able to control and/or reduce the effects of a Chlamydia infection. The antibiotic will be provided under the form of a drug to be taken orally.

The pharmaceutical composition of the invention is not proposed for a systematic prevention of atherosclerosis, but is proposed for long-time (years) treatment of patients suffering from atherosclerosis (in coronaries, carotids, cerebral vessels) preferably applicable either after a vascular event (cerebral or coronary thrombosis), either to avoid recurrences after stenting or coronary artery bypass, or to avoid the extension of atherosclerotic lesions in the carotid arteries.

The treatment can also be used to prevent re-occurrence of atheroma after cardiac bypass surgery and/or after stenting; as preventive agent in the high risks states, such as severe hypercholesterolemia; and to avoid diabetic vascular complications.

When the micro-organism (Chlamydia) is detected in a patient suffering from atherosclerosis (coronary, carotids, cerebral vessels) (detected following several clinical examinations and biological parameters: ECG, echography, coronography, plasma value of C-reactive protein) and when serum analysis and polymerase-chain reaction analysis have demonstrated, at least once, the presence of (anti-chlamydia) antibodies or (chlamydial) DNA, the pharmaceutical composition of the invention is administrated to the patient for a long (years) duration, preferably with a recurrent antibiotherapy with macrolides. The treatment will be as follows
1. antibiotic (macrolide) administration for ten days, together with the pharmaceutical composition of the invention.
2. administration of the pharmaceutical composition of the invention for six weeks.
3. a new treatment of ten days with the antibiotic together with the pharmaceutical composition of the invention.
4. administration of the pharmaceutical composition of the invention alone for a new six weeks, followed by an new period of ten days with the antibiotic, etc.

In the pharmaceutical composition of the invention, a glucocorticoid is added when inflammatory phenomena are detected as demonstrated by a positive blood C-reactive protein (CRP) value, and is continued until the return of CRP to normal blood value (an abnormal state of muscle fatigue is an indication for blood CRP concentration measurement). This alternate treatment is to be continued for months or years, depending from one patient to another. As Chlamydia pneumoniae is intracellular and cannot be eradicated by antibiotic treatment, the pharmaceutical composition of the invention is proposed to avoid a continuous treatment by antibiotics and the treatment is to be administered for years (at least until the discovery of an antibiotic that should eradicate the microorganism: this kind of antibiotic is still unknown).

The pharmaceutical composition could also be administrated to a patient after a vascular event (stroke or arterial thrombosis), for recurrence after stenting or coronary bypass, to prevent an extension of carotid atherosclerotic lesions when these arterial lesions have been evidenced, or after surgical curettage of the arteries, even if intracellular micro-organisms have not been evidenced in these patients In these groups of patients, three-months treatments with antibiotics (for example the ROXIS study) have demonstrated a slowering of atherosclerosis progression, but the slowering was of short duration. A treatment with the pharmaceutical composition is thus recommended for months and years in these patients.

The doses of the corticosteroids used in the pharmaceutical composition are calculated not to reach immunosuppression (in order to preserve the defence response to other pathogenic micro-organisms), but to modulate NADPH-oxidase activity and expression, to inhibit an excessive production of oxidant species, and to decrease the inflammation response especially the cytokine (TNFα, IL8) production and the expression of adhesion molecules on leucocytes.

As mentioned above, when the micro-organism (Chlamydia) can be characterised by genetic or serological analysis, the proposed treatment should be completed by recurrent short-time treatments with macrolide antibiotics or other therapeutical compounds (antiviral active ingredients possibly present in the pharmaceutical composition of the invention), to which the micro-organism (Chlamydia) is highly sensitive.

Another aspect of the present invention is related to the use of the pharmaceutical composition according to the invention for the manufacture of a medicament to be used in the treatment and/or the prevention of atherosclerosis, in particular atherosclerosis from infectious origin, more particularly atherosclerosis induced by an intracellular micro-organism, especially Chlamydia pneumoniae.

Another aspect of the present invention is related to a method of treatment of a mammal patient including a human patient, which comprises the step of administrating a sufficient amount of the pharmaceutical composition according to the invention in order to treat and/or to prevent atherosclerosis in said mammal patient, especially atherosclerosis from infectious origin, more particularly atherosclerosis induced by Chlamydia pneumoniae.

With an <<effective amount>>, a <<therapeutically effective amount>> or a <<sufficient amount>> in the present context is meant a non-toxic but sufficient amount of the agent, active compound or ingredient to provide the desired therapeutic effect. The exact amount that is required herefor will vary from subject to subject, depending on the species, age, and general condition of the subject, mode of administration and the like. An appropriate <<effective amount>> may be determined by one of skill in the art using only routine experimentation.

The following therapeutic regimens are of particular interest:
the administration to a subject in need thereof of one of the compositions of the invention, in particular one that comprises a polyphenol (flavonoid) as stabilizing agent, over a long period. With a <<long period>> in the present context is meant a period of at least months/years, preferably at least one year, most preferably at least several years.
the above treatment whereby the composition is administered over a period of years, but cut by periodic arrests of several weeks.
the above-proposed treatment(s) may be combined with a separate antibiotic treatment, for instance a treatment with macrolides. The antibiotic treatment may be a continuous treatment, but preferably is one with periodic arrests. Preferably a composition according to the invention is then administered continuously, together with the antibiotic treatment and during the period of periodic arrest of antibiotics

As mentioned above, the compositions according to the invention are preferably administered in a transcutaneous way.

### SHORT DESCRIPTION OF THE DRAWINGS

FIG. 1 represents the transformation of monocytes (1) into macrophages (2) under the effect of an overnight (19 hours) incubation with Chlamydia pneumoniae.

FIG. 2 represents the increase of the nitrate (part 1) and hydrogen peroxide (part 2) production by THP-1 cells conditioned (pre-incubated for 19 hours) with Chlamydia pneumoniae, and then stimulated by PMA (phorbol-12-myristate-13-acetate) 10⁻⁷ M. Nitrates: nanomoles/10⁶ cells; hydrogen peroxide: picomoles/10⁶ cells.

| Column nr | Column identification |
|---|---|
| 1 | THP-1 |
| 2 | THP-1 + Chlamydia |
| 3 | THO-1 + Chlamydia + PMA 10⁻⁷ M |

FIG. 3 represents ethylene production (expressed in picomoles/10⁶ cells) by THP-1 cells conditioned with Chlamydia pneumoniae, and then stimulated by PMA 10⁻⁷ M (column 3); effects of superoxide dismutase (SOD 100 U/ml), inhibitor of superoxide anion (column 4); effects of L-N-monomethyl arginine (L-NMMA 100 and 10 μM), inhibitor of NOsynthase (columns 5 and 6); and effects of diphenyl iodonium (DPI 5 μM), inhibitor of NADPH-oxidase and NOsynthase (column 7). Columns 1 and 2 are controls respectively with TPH-1 alone and THP-1 conditioned with C. pneumoniae but not stimulated with PMA.

FIG. 4 represents oxygen consumption (in micromoles) by monocytes (THP-1 cells) before and after their stimulation with PMA 5×10⁻⁷ M (added after 15 minutes: double headed arrow on the figure). Curve 1: THP-1 cells conditioned by incubation with C pneumoniae. Curve 2: THP-1 cells without preconditioning with C pneumoniae. On the abscissa: time in minutes.

FIG. 5 represents electronic paramagnetic 5 resonance (EPR) demonstration of the production of superoxide anion by PMA (5×10⁻⁷M) stimulated THP-1 cells, which have been conditioned with Chlamydia (Mouithys-Mickalad et al., 2001, Biochem Biophys Res Commun 287(3) :781-788)

| I. EPR with spin trap DMPO 100 mM | |
|---|---|
| 1. | THP-1 + Chlamydia (no PMA) |
| 2. | THP-1 + PMA (no Chlamydia) |
| 3. | THP-1 + Chlamydia + PMA |
| 4. | same as 3 + 200 U SOD |
| 5. | same as 3 + 10 μM DPI |
| II. EPR with spin trap DEPMPO 10 mM | |
| 1. | THP-1 + PMA (no Chlamydia) |
| 2. | THP-1 + Chlamydia + PMA |
| 3. | same 2 + 200 U SOD |
| 4. | same as 2 + 10 μM DPI |

FIG. 6 represents the effects of pre-incubation (19 hours) of THP-1 with Chlamydia on the production of cytokines TNFα (part 1) and IL-8 (part 2) (measured in the culture supernatants). TNFα is expressed in picogrammes/ml and IL-8 in nanogrammes/ml.

Column 1: THP-1 alone; column 2: THP-1+Chlamydia

FIG. 7 represents the effects of pre-incubation with Chlamydia on the activity of the nuclear transcription factor kappaB (NF-kB) (column 2). NF-kB is expressed in % of column 1 (THP-1 cells without preincubation with C pneumoniae)

| Column | | NF-κB relative |
|---|---|---|
| nr | Column identification | value (%) |
| 1 | THP-1 | 100 |
| 2 | THP-1 + Chlamydia | 405 |
| 3 | THP-1 + Chlamydia + HCT 10⁻⁵ M | 396 |
| 4 | THP-1 + Chlamydia + MPL 10⁻⁵ M | 417 |
| 5 | THP-1 + Chlamydia + HCT 10⁻⁶ M | 480 |
| 6 | THP-1 + Chlamydia + MPL 10⁻⁶ M | 450 |

HCT: hydrocortisone;
MPL: methylprednisolone

FIG. 8 represents the effects of pre-incubation (19 hours) with Chlamydia pneumoniae on the gene expression (relative values) by THP-1 cells. The studied genes are IL-1β (part 1), IL-6 (part 2), IL-8 (part 3), COX-2 (part 4) and a subunit of NADPH-oxidase p₂₂^{phox} (part 5).

THP-1 Incubated with Chlamydia (Column 2) are Compared to THP-1 without Chlamydia (Column 1).

FIG. 9 represents the effects of hydrocortisone (HCT) on the oxidant activity of THP-1 cells conditioned either with Chlamydia pneumoniae (part 1; n=13) or with Escherichia Coli endotoxin (LPS) (part 2), and then stimuled by PMA 10⁻⁷ M. Ethylene is expressed in % of the value measured for THP-1 cells incubated with Chlamydia (column 2 for part 1), or incubated with LPS (column 1 for par 2).

| Column | Part 1 | | Part 2 | |
|---|---|---|---|---|
| nr | Identification Ethylene | | Identification Ethylene | |
| 1 | THP-1 | 38 | THP-1 + LPS | 100 |
| 2 | THP-1 + Chlamydia | 100 | THP-1 + LPS + HCT | 19 |
| | | | 10⁻⁴ M | |
| 3 | THP-1 + Chlamydia + | 108 | THP-1 + LPS + HCT | 50 |
| | HCT 10⁻⁴ M | | 10⁻⁵ M | |
| 4 | THP-1 + Chlamydia + | 119 | THP-1 + LPS + HCT | 57 |
| | HCT 10⁻⁵ M | | 10⁻⁶ M | |
| 5 | THP-1 + Chlamydia + | 139 | | |
| | HCT 10⁻⁶ M | | | |

FIG. 10 represents the effects of estradiol on the oxidant activity of THP-1 cells conditioned with Chlamydia and then stimulated by 10⁻⁷ M PMA. Estradiol is incubated together with Chlamydia (columns 3, 4 and 5) or added just before activation by PMA (columns 6, 7 and 8). Ethylene value are expressed in % of the value measured for THP-1 cells incubated with Chlamydia (column 2).

| Column | Column identification | Ethylene |
|---|---|---|
| 1 | THP-1 | 72.0 |
| 2 | THP-1 + Chlamydia | 100.0 |
| 3 | THP-1 + Chlamydia + [symbol] stradiol pre-incubated | 140.0 |
| | 10⁻⁴ M | |
| 4 | THP-1 + Chlamydia + [symbol] stradiol pre-incubated | 133.0 |
| | 10⁻⁵ M | |
| 5 | THP-1 + Chlamydia + [symbol] stradiol pre-incubated | 85.0 |
| | 10⁻⁶ M | |
| 6 | THP-1 + Chlamydia + [symbol] stradiol not pre-incubated | 2.6 |
| | 10⁻⁴ M | |
| 7 | THP-1 + Chlamydia + [symbol] stradiol not pre-incubated | 2.9 |
| | 10⁻⁵ M | |
| 8 | THP-1 + Chlamydia + [symbol] stradiol not pre-incubated | 7.8 |
| | 10⁻⁶ M | |

FIG. 11 represents the effects of tocopherol (vitamin E) on the oxidant activity of THP-1 cells conditioned with Chlamydia and then stimulated by 10⁻⁷ M PMA. Tocopherol is incubated together with Chlamydia (columns 4, 5 and 6) or added just before activation by PMA (columns 7, 8 and 9) Ethylene value are expressed in % of the value measured for THP-1 cells incubated with Chlamydia (column 3).

| Column | | |
|---|---|---|
| nr | Column identification | Ethylene |
| 1 | THP-1 | 35.0 |
| 2 | Chlamydia | 0.0 |
| 3 | THP-1 + Chlamydia | 100.0 |
| 4 | THP-1 + Chlamydia + Tocopherol 10⁻⁴ M | 147.0 |
| | pre-incubated | |
| 5 | THP-1 + Chlamydia + Tocopherol 10⁻⁶ M | 133.0 |
| | pre-incubated | |
| 6 | THP-1 + Chlamydia + Tocopherol 10⁻⁶ M | 87.0 |
| | pre-incubated | |
| 7 | THP-1 + Chlamydia + Tocopherol 10⁻⁴ M | 2.4 |
| | not pre-incubated | |
| 8 | THP-1 + Chlamydia + Tocopherol 10⁻⁵ M | 2.5 |
| | not pre-incubated | |
| 9 | THP-1 + Chlamydia + Tocopherol 10⁻⁶ M | 77.0 |
| | not pre-incubated | |

FIG. 12 represents the effects of quercetin (formula in insert) on the oxidant activity of THP-1 cells conditioned with Chlamydia and then stimulated by 10⁻⁷ M PMA. Quercetin is incubated together with Chlamydia (columns 4, 5 and 6) or added just before activation by PMA (columns 7, 8 and 9). Ethylene value are expressed in % of the value measured for THP-1 cells incubated with Chlamydia (column 3)

| Column | | |
|---|---|---|
| nr | Column identification | Ethylene |
| 1 | THP-1 + Chlamydia | 72.2 |
| 2 | Chlamydia | 0.0 |
| 3 | THP-1 + Chlamydia | 100.0 |
| 4 | THP-1 + Chlamydia + Quercetin 10⁻⁴ M incubated | 2.0 |
| 5 | THP-1 + Chlamydia + Quercetin 10⁻⁵ M incubated | 100.0 |
| 6 | THP-1 + Chlamydia + Quercetin 10⁻⁶ M incubated | 131.0 |
| 7 | THP-1 + Chlamydia + Quercetin 10⁻⁴ M not | 3.7 |
| | incubated | |
| 8 | THP-1 + Chlamydia + Quercetin 10⁻⁵ M not | 1.2 |
| | incubated | |
| 9 | THP-1 + Chlamydia + Quercetine 10⁻⁶ M not | 6.6 |
| | incubated | |

FIG. 13 represents the effects of hydrocortisone (HCT) on the production of TNFα (part 1) and IL-8 (part 2) by THP-1 cells pre-incubated (19h) with Chlamydia pneumoniae. TNFα is expressed in picogrammes/ml and IL-8 in nanogrammes/ml.

| Column nr | Column identification |
|---|---|
| 1 | THP-1 |
| 2 | THP-1 + Chlamydia |
| 3 | THP-1 + Chlamydia + HCT 10⁻⁹ M |
| 4 | THP-1 + Chlamydia + HCT 10⁻⁸ M |
| 5 | THP-1 + Chlamydia + HCT 10⁻⁷ M |
| 6 | THP-1 + Chlamydia + HCT 10⁻⁶ M |
| 7 | THP-1 + Chlamydia + HCT 10⁻⁵ M |

FIG. 14 represents the effects of methylprednisolone (MPL) on the oxidant activity of THP-1 cells conditioned with Chlamydia pneumoniae and then activated by 10⁻⁷ M PMA. MPL is incubated together with Chlamydia (black columns) or added just before activation by PMA (grey columns). The ethylene values are expressed in % of the value measured for THP-1 cells incubated with Chlamydia (column 2).

| Column nr | Column identification |
|---|---|
| 1 | THP-1 |
| 2 | THP-1 + Chlamydia |
| 3 | THP-1 + Chlamydia + MPL 10⁻⁴ M incubated |
| 4 | THP-1 + Chlamydia + MPL 10⁻⁴ M not incubated |
| 5 | THP-1 + Chlamydia + MPL 10⁻⁵ M incubated |
| 6 | THP-1 + Chlamydia + MPL 10⁻⁵ M not incubated |
| 7 | THP-1 + Chlamydia + MPL 10⁻⁶ M incubated |
| 8 | THP-1 + Chlamydia + MPL 10⁻⁶ M not incubated |

FIG. 15 represents the effects of methylprednisolone (MPL) on the production of TNFα (part 1) and IL-8 (part 2) by THP-1 cells pre-incubated with Chlamydia pneumoniae. TNFα is expressed in picogrammes/ml and IL-8 in nanogrammes/ml.

| Column nr | Column identification |
|---|---|
| 1 | THP-1 |
| 2 | THP-1 + Chlamydia |
| 3 | THP-1 + Chlamydia + MPL 10⁻⁹ M |
| 4 | THP-1 + Chlamydia + MPL 10⁻⁸ M |
| 5 | THP-1 + Chlamydia + MPL 10⁻⁷ M |
| 6 | THP-1 + Chlamydia + MPL 10⁻⁶ M |
| 7 | THP-1 + Chlamydia + MPL 10⁻⁵ M |

FIG. 16 represents the genetic expression of one of the subunits of NADPH-oxidase, the p₂₂^{phox}, and the effects of glucocorticoids, hydrocortisone (HCT) and methylprednisolone (MPL) (data are expressed as relative values by comparison with a reference gene).

| Column nr | Column identification |
|---|---|
| 1 | THP-1 |
| 2 | THP-1 + Chlamydia |
| 3 | THP-1 + Chlamydia + HCT 10⁻⁵ M |
| 4 | THP-1 + Chlamydia + HCT 10⁻⁶ M |
| 5 | THP-1 + Chlamydia + MPL 10⁻⁴ M |
| 6 | THP-1 + Chlamydia + MPL 10⁻⁵ M |
| 7 | THP-1 + Chlamydia + MPL 10⁻⁶ M |

FIG. 17 represents the chemical structures of the stilbene molecules used in the THP-1 cell model.
1.1. trans-reveratrol; 2. piceatanol (3,4,3′,5′tetrahydrostilbene); 3.trans-4-hydrostilbene; 4. bertrol (trans-α-α-diethyl-p,p′-stilbenediol). The compounds 3 and 4 are carcinogenic.

FIG. 18 represents the effects of resveratrol on the oxidation rate of THP-1 cells conditioned with Chlamydia pneumoniae and then stimulated by 10⁻⁷ M PMA. Resveratrol is incubated together with Chlamydia (black columns) or added just before activation by PMA (grey columns). The ethylene values are expressed in % of the value measured for THP-1 cells incubated with Chlamydia (column 2).

| Column nr | Column identification |
|---|---|
| 1 | THP-1 |
| 2 | THP-1 + Chlamydia |
| 3 | THP-1 + Chlamydia + resveratrol 10⁻⁴ M incubated |
| 4 | THP-1 + Chlamydia + resveratrol 10⁻⁴ M not incubated |
| 5 | THP-1 + Chlamydia + resveratrol 10⁻⁵ M incubated |
| 6 | THP-1 + Chlamydia + resveratrol 10⁻⁵ M not incubated |
| 7 | THP-1 + Chlamydia + resveratrol 10⁻⁶ M incubated |
| 8 | THP-1 + Chlamydia + resveratrol 10⁻⁶ M not incubated |

FIG. 19 represents the effects of the association hydrocortisone (HCT)/resveratrol on the oxidation rate of THP-1 cells conditioned with Chlamydia pneumoniae. The ethylene values are expressed in % of the value measured for THP-1 cells incubated with Chlamydia (column 3).

| Column | | |
|---|---|---|
| nr | | Ethylene |
| 1 | THP-1 | 71.0 |
| 2 | Chlamydia | 0.0 |
| 3 | THP-1 + Chlamydia | 100.0 |
| 4 | THP-1 + Chlamydia + Resveratrol 10⁻⁴ M | 18.3 |
| 5 | THP-1 + Chlamydia + Resveratrol 10⁻⁵ M | 35.8 |
| 6 | THP-1 + Chlamydia + HCT 10⁻⁴ M | 84.0 |
| 7 | THP-1 + Chlamydia + HCT 10⁻⁵ M | 116.0 |
| 8 | THP-1 + Chlamydia + HCT 10⁻⁴ M + Resveratrol | 53.0 |
| | 10⁻⁴ M | |
| 9 | THP-1 + Chlamydia + HCT 10⁻⁴ M + Resveratrol | 52.0 |
| | 10⁻⁵ M | |
| 10 | THP-1 + Chlamydia + HCT 10⁻⁵ M + Resveratrol | 41.0 |
| | 10⁻⁴ M | |
| 11 | THP-1 + Chlamydia + HCT 10⁻⁵ M + Resveratrol | 87.0 |
| | 10⁻⁵ M | |

FIG. 20 represents the effects of the association hydrocortisone (HCT)/piceatanol on the oxidation rate of THP-1 cells conditioned with Chlamydia pneumoniae. The ethylene values are expressed in % of the value measured for THP-1 cells incubated with Chlamydia (column 3).

| Column | | |
|---|---|---|
| nr | | Ethylene |
| 1 | THP-1 | 32.5 |
| 2 | Chlamydia | 0.0 |
| 3 | THP-1 + Chlamydia | 100.0 |
| 4 | THP-1 + Chlamydia + piceatanol 10⁻⁴ M | 0.4 |
| 5 | THP-1 + Chlamydia + piceatanol 10⁻⁵ M | 57.0 |
| 6 | THP-1 + Chlamydia + HCT 10⁻⁴ M | 98.0 |
| 7 | THP-1 + Chlamydia + HCT 10⁻⁵ M | 85.0 |
| 8 | THP-1 + Chlamydia + HCT 10⁻⁴ M + piceatanol | 1.4 |
| | 10⁻⁴ M | |
| 9 | THP-1 + Chlamydia + HCT 10⁻⁴ M + piceatanol | 73.2 |
| | 10⁻⁵ M | |
| 10 | THP-1 + Chlamydia + HCT 10⁻⁵ M + piceatanol | 0.6 |
| | 10⁻⁴ M | |
| 11 | THP-1 + Chlamydia + HCT 10⁻⁵ M + piceatanol | 35.4 |
| | 10⁻⁵ M | |

FIG. 21 represents the effects of the association hydrocortisone (HCT)/bertrol (trans-α-α-yl-p,p′-stilbenediol) on the oxidation rate of THP-1 conditioned with Chlamydia pneumoniae. The ethylene s are expressed in % of the value measured for THP-1 incubated with Chlamydia (column 3).

| Column | | |
|---|---|---|
| nr | | Ethylene |
| 1 | THP-1 | 10.5 |
| 2 | Chlamydia | 0.0 |
| 3 | THP-1 + Chlamydia | 100.0 |
| 4 | THP-1 + Chlamydia + bertrol 10⁻⁴ M | 1.6 |
| 5 | THP-1 + Chlamydia + bertrol 10⁻⁵ M | 32.0 |
| 6 | THP-1 + Chlamydia + HCT 10⁻⁴ M | 104.0 |
| 7 | THP-1 + Chlamydia + HCT 10⁻⁵ M | 72.0 |
| 8 | THP-1 + Chlamydia + HCT 10⁻⁴ M + bertrol | 3.1 |
| | 10⁻⁴ M | |
| 9 | THP-1 + Chlamydia + HCT 10⁻⁴ M + bertrol | 46.4 |
| | 10⁻⁵ M | |
| 10 | THP-1 + Chlamydia + HCT 10⁻⁵ M + bertrol | 2.0 |
| | 10⁻⁴ M | |
| 11 | THP-1 + Chlamydia + HCT 10⁻⁵ M + bertrol | 17.4 |
| | 10⁻⁵ M | |

FIG. 22 represents the effects of the association hydrocortisone (HCT)/trans-4-stilbene on the oxidation rate of THP-1 cells conditioned with Chlamydia pneumoniae. The ethylene values are expressed in % of the value measured for THP-1 cells incubated with Chlamydia (column 3).

| Column | | |
|---|---|---|
| nr | | Ethylene |
| 1 | THP-1 | 18.0 |
| 2 | Chlamydia | 0.0 |
| 3 | THP-1 + Chlamydia | 100.0 |
| 4 | THP-1 + Chlamydia + trans-4-stilbene 10⁻⁴ M | 1.8 |
| 5 | THP-1 + Chlamydia + trans-4-stilbene 10⁻⁵ M | 14.0 |
| 6 | THP-1 + Chlamydia + HCT 10⁻⁴ M | 118.0 |
| 7 | THP-1 + Chlamydia + HCT 10⁻⁵ M | 68.1 |
| 8 | THP-1 + Chlamydia + HCT 10⁻⁴ M + trans-4- | 1.5 |
| | stilbene 10⁻⁴ M | |
| 9 | THP-1 + Chlamydia + HCT 10⁻⁴ M + trans-4- | 64.0 |
| | stilbene 10⁻⁵ M | |
| 10 | THP-1 + Chlamydia + HCT 10⁻⁵ M + trans-4- | 2.5 |
| | stilbene 10⁻⁴ M | |
| 11 | THP-1 + Chlamydia + HCT 10⁻⁵ M + trans-4- | 49.0 |
| | stilbene 10⁻⁵ M | |

FIG. 23 represents the combined action of a glucocorticoid (hydrocortisone) and resveratrol on the production of the cytokines TNFα (part 1) and IL-8 (part 2) by THP-1 cells incubated (19 hours) with Chlamydia pneumoniae. TNFα is expressed in picogrammes/ml and IL-8 in nanogrammes/ml.

| Column nr | Column identification |
|---|---|
| 1 | THP-1 |
| 2 | THP-1 + Chlamydia |
| 3 | THP-1 + Chlamydia + hydrocortisone 10⁻⁶ M + resveratrol |
| | 10⁻⁵ M |

FIG. 24 represents the combined action of resveratrol and a flavonoid (rutin or quercetin) on the oxidation rate of THP-1 cells conditioned with Chlamydia pneumoniae. The ethylene values are expressed in % of the value masured for THP-1 cells incubated with Chlamydia (column 2).

| Colum nr | Column identification |
|---|---|
| 1 | THP-1 |
| 2 | THP-1 + Chlamydia |
| 3 | THP-1 + Chlamydia + resveratrol 10⁻⁴ M |
| 4 | THP-1 + Chlamydia + resveratrol 10⁻⁵ M |
| 5 | THP-1 + Chlamydia + resveratrol 10⁻⁶ M |
| 6 | THP-1 + Chlamydia + rutin 10⁻⁶ M |
| 7 | THP-1 + Chlamydia + quercetin 10⁻⁶ M |
| 8 | THP-1 + Chlamydia + resveratrol 10⁻⁴ M + rutin 10⁻⁶ M |
| 9 | THP-1 + Chlamydia + resveratrol 10⁻⁴ M + quercetin 10⁻⁶ M |
| 10 | THP-1 + Chlamydia + resveratrol 10⁻⁵ M + rutin 10⁻⁴ M |
| 11 | THP-1 + Chlamydia + resveratrol 10⁻⁵ M + rutin 10⁻⁵ M |
| 12 | THP-1 + Chlamydia + resveratrol 10⁻⁵ M + rutin 10⁻⁶ M |
| 13 | THP-1 + Chlamydia + resveratrol 10⁻⁵ M + quercetin 10⁻⁶ M |
| 14 | THP-1 + Chlamydia + resveratrol 10⁻⁶ M + rutin 10⁻⁴ M |
| 15 | THP-1 + Chlamydia + resveratrol 10⁻⁶ M + rutin 10⁻⁵ M |
| 16 | THP-1 + Chlamydia + resveratrol 10⁻⁶ M + rutin 10⁻⁶ M |
| 17 | THP-1 + Chlamydia + resveratrol 10⁻⁶ M + quercetin 10⁻⁶ M |
| 18 | THP-1 + Chlamydia + resveratrol 10⁻⁷ M + rutin 10⁻⁷ M |
| 19 | THP-1 + Chlamydia + resveratrol 10⁻⁷ M + quercetin 10⁻⁷ M |

FIG. 25 represents the model of monocyte transformation into macrophages (1) and then into foam cells (2) in the presence of low density lipoproteins, as well as the effects of 10⁻⁵M and 10⁻⁶M hydrocortisone (3 and 4), 10⁻⁵M resveratrol (5) and the association 10⁻⁵M hydrocortisone/10⁻⁵M resveratol (6) on the formation of foam cells. Arrows indicate lipid vesicles stained with Oil Red O.

FIG. 26 represents the model of monocyte transformation into macrophages (1) and into foam cells (2) in the presence of liposomes, with the effects of the association of 10⁻⁶ M resveratrol and 10⁻⁶M rutin (2). Arrows indicate lipid vesicles stained with Oil Red O.

The present invention will be described in more details in the following non-limiting description of preferred embodiments of the present invention, in reference to the enclosed figures.

### DETAILED DESCRIPTION

Description of the Model Used for Evidence of Chlamydia pneumoniae Effects on the Cellular Metabolism

The model consists in the culture of the human monocytes (THP-1 cell line), in which the production of oxidant species is measured by accurate techniques, which avoid artefacts:
gas-liquid chromatography
electron paramagnetic resonance (EPR) for unequivocal demonstration of superoxide anion production.
   This model has been described in details in Mouithys Mickalad et al. (Biochem Biophys Res Comm 2001, 287: 781-788).
   Treatment of the Cells with Chlamydia pneumoniae:

The monocytes (in multiwell plates, 2×10⁶ cells/well) are conditioned by a pre-incubation of 19 hours with elementary bodies of Chlamydia pneumoniae (at a dose equivalent to a mean endotoxin concentration of 3.3 pg). The elementary bodies are obtained by Chlamydia culture in MacCoy cells (American Type Culture Collection, Rockville, USA).

Measurement of Oxidative Metabolism:

After incubation with C. pneumoniae, the cells are washed and detached from the wells; they are put in sterile vials and an oxidable substrate, α-keto-methyl butyric acid (KMB) at 10⁻³ M, is added. The vial is sealed and the following reagents are added by needle puncture. through the septum: 200 U horseradish peroxidase (HRP) and 10⁻⁷ M phorbol myristate acetate (PMA).

PMA is a monocyte activator, enhancing the superoxide anion production by these cells. Superoxide anion dismutates into hydrogen peroxide (H₂O₂), which is used by HRP to form more oxidant species, able to oxidize KMB, releasing ethylene. After 3 hours of incubation at 37° C, ethylene, which has accumulated in the gaseous phase of the sealed vial is measured by gas liquid chromatography, on a Porapak T column (TM) equipped with flame ionisation detector. These assays can be made with the prior addition of a reagent able to inhibit or to stimulate the monocytes, the reagent being added, either at pre-incubation (at the same time as the Chlamydia EB), or at the moment of PMA activation.

Superoxide Anion Production (Radicalar Species)

Monocytes are conditioned by Chlamydia. The formation of this species was monitored by electron paramagnetic resonance, using spin-trapping agents.

Checking of H₂O₂ Formation:

H₂O₂ formation was verified by a spectrophotometric method (using isothiocyanate).

Oxygen Consumption:

Oxygen consumption which is induced by monocyte stimulation and due to NADPH-oxidase activity was measured by oxymetry (Clark electrode (TM)(Oxygraph OROBOROS, Grinzens, Austria).

NO Synthase Activation:

The production of nitric oxide (NO) was checked by nitrate measurement (Griess technique,. Green et al., 1982, Anal Biochem. 126: 131-138. using nitrate reductase).

A Conversion of Monocytes into Macrophages

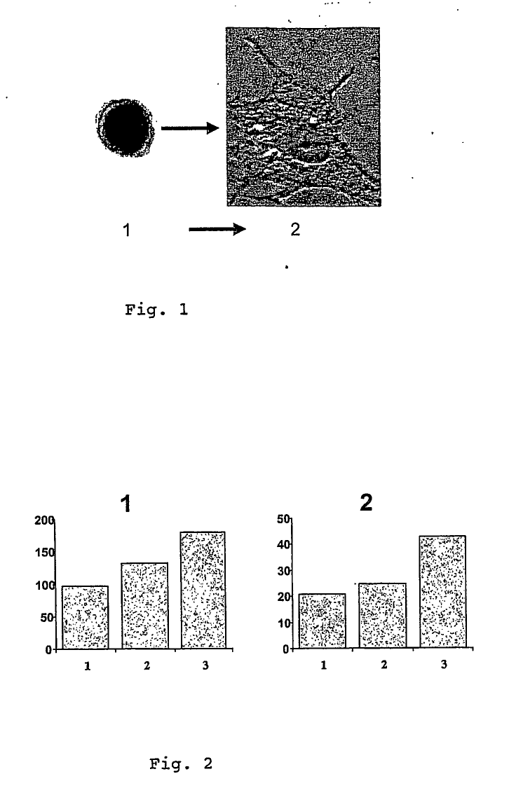

Incubation of THP-1 cells with C. pneumoniae induces the differentiation of monocytes (round floating cells, 10⁻¹⁵ μm diameter; FIG. 1, 1) into macrophages, amiboid cells, which are larger than monocytes (30-50 μm diameter) and adhere to the support (FIGS. 1,2). Monocytes are stained with Giemsa and May-Grunwald stains (Merck, Germany) (Chemical methods of medical investigation, 1964, 10^{th} edition by E. Merck AG. Darmstadt, Germany) and macrophages are observed by phase contrast microscopy.

Stimulation of the Oxidant Metabolism, Mainly Via the NADPH-Oxidase Pathway

The monocytes are firstly conditioned during 19 hours in the presence of C. pneumoniae and then stimulated by PMA. These monocytes produce more nitrates (FIG. 2 part 1, column 3) and H₂O₂ (FIG. 2 part 2, column 3) than monocytes conditioned with Chlamydia, but without PMA stimulation (columns 2 part 1 and part 2), and more than monocytes not conditioned and not stimulated by PMA (columns 1 of parts 1 and 2).

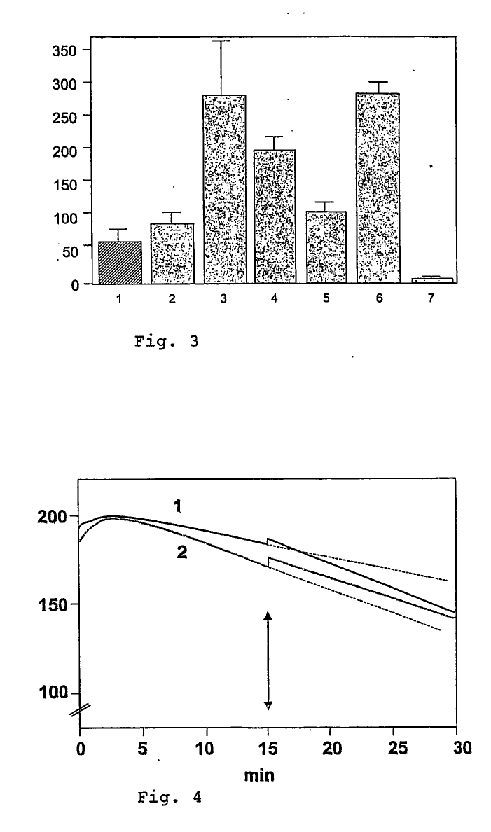

Ethylene production (obtained by oxidation of KMB) is enhanced in the case of Chlamydia-conditioned THP-1 (FIG. 3, column 2) compared to PMA-stimulated monocytes but not conditioned by Chlamydia (FIG. 3, column 1). But the ethylene production is particularly enhanced when the monocytes are stimulated by PMA after a 19 hours conditioning with Chlamydia (FIG. 3, column 3). This increase of KMB oxidation rate is explained by the action of the oxidant species produced from H₂O₂ by HRP, and by the activity of peroxynitrite formed in situ by the reaction of superoxide anion with NO.

This stimulation of the oxidative metabolism of monocytes conditioned by C. pneumoniae is confirmed by the measurement of oxygen consumption. When the Chlamydia-conditioned cells are stimulated by PMA, an increase in the slope of the curve of oxygen consumption (FIG. 4, curve 1) is observed compared to the cells not conditioned by C. pneumoniae (FIG. 4, curve 2).

The curve 2 is obtained with monocytes alone and the curve 1 with monocytes pre-incubated with Chlamydia. The figure shows that the addition of PMA accelerates the consumption of oxygen by cells conditioned with Chlamydia (increase of the slope of the curve 1).

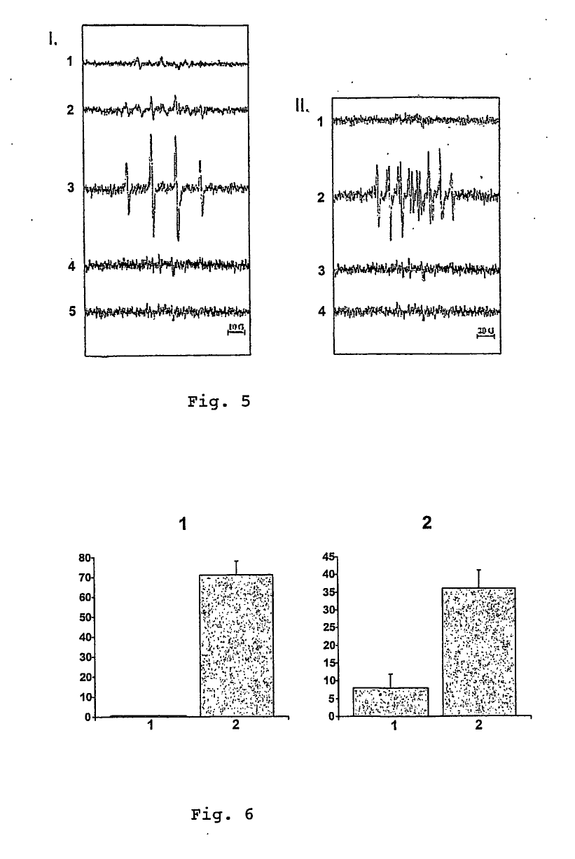

The electron paramagnetic resonance analysis (FIG. 5) demonstrates that the monocytes conditioned with C. pneumoniae and then stimulated by PMA produce superoxide anion.

Part I: EPR spectra obtained with the spin trap DMPO: the spectrum is characteristic of the radical spin adduct DMPO-OH (line 3).

Part II: EPR spectra obtained with the spin trap DEPMPO: the spectrum is characteristic of the radical spin adduct DEPMPO-OOH (line 2).

The addition of superoxide dismutase (SOD) or diphenyl iodonium (DPI) decreases the EPR signal intensity (lines I 4 and 5, and lines II 3 and 4).

The line I-1 is a control spectrum obtained with cells pre-incubated with Chlamydia, but without PMA stimulation. The lines I-2 and II-1 are control spectra obtained with the cells stimulated with PMA, but without pre-incubation with Chlamydia.

With DMPO, one observes a four line spectrum of high intensity, characteristic of the spin adduct DMPO-OH (FIG. 5-I, line 3). With DEPMPO, the more complex spectrum of the spin adduct DEPMPO-OOH, characteristic of superoxide anion is observed (FIG. 5-II, line 2). The lines 1 and 2 on the FIG. 5-I and the line 1 on the FIG. 5-II are control spectra obtained with cells not stimulated by PMA (I-1) or cells stimulated by PMA but non conditioned with Chlamydia (I-2 et II-1).

Confirmation of the Role of NADPH-Oxidase and NO Synthase:

Superoxide anion formation, followed by its dismutation into H₂O₂, implicates the activity of NADPH-oxidase, and the nitrate production implicates the activity of NO synthase. The role played by these enzymes in the synthesis of superoxide anion, H₂O₂ and nitrates, is confirmed by utilisation of:
an inhibitor of NADPH-oxidase and NO synthase, diphenyliodonium (DPI): DPI inhibits almost totally the ethylene production (FIG. 3, column 7) and suppresses the EPR signal of superoxide anion (FIG. 5, lines I-5 and II-4).
superoxide dismutase (SOD), which accelerates the superoxide anion dismutation into H₂O₂. By preventing the formation of peroxynitrite, SOD reduces by ±30% the ethylene production, confirming the major role of H₂O₂ and the partial role of peroxynitrite in the oxidation of KMB (FIG. 3, column 4). In EPR, SOD suppresses almost totally the superoxide anion signal (FIG. 5, lines I-4 and II-3).
L-NMMA [L-N monomethyl argininel, an inhibitor of NO synthase: at 10-4 M (100 μM), it reduces by 60% the ethylene formation, but it is without effect at 10⁻⁵ M (10 μM), confirming the partial role of peroxynitrite formed in situ (FIG. 3, columns 5 and 6).
   Activation of Cytokines Production

By using this model, one may verify that the monocyte incubation with C. pneumoniae during 19 hours, enhances the production of TNFα(FIG. 6, part 1) and IL-8 (FIG. 5, part 2), as measured by immunological methods (ELISA type) in the culture supernatant.

Activation of the Nuclear Transcription Factor kappaB (NF-κB)

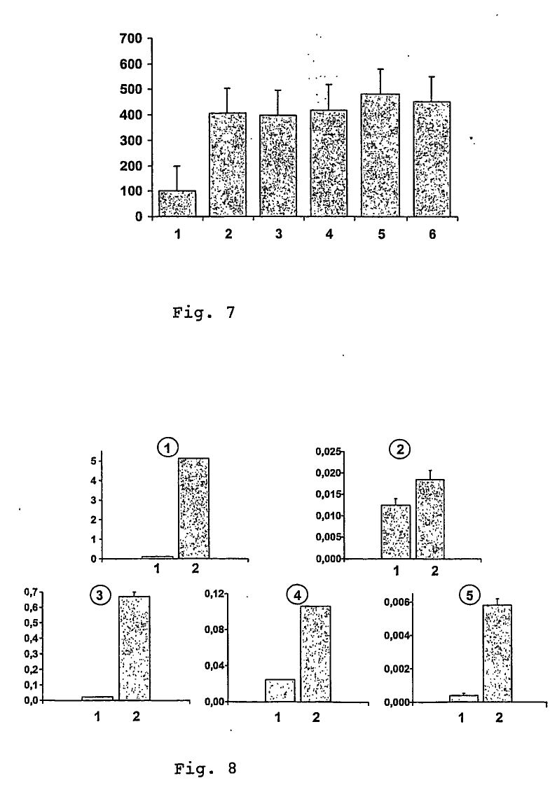

The activation of NF-κB was measured by the technique of <<electrophoretic mobility shift assay>> (EMSA) (Schoonbroodt et al., 2000, J Immunol. 164: 4292-4300; Nys et al., 2003, Nitric Oxide 9: 33-43) (FIG. 7). The activation of NF-κB is expressed in % of control cells (cells which have not been pre-incubated with Chlamydia) (column 1). The incubation of monocytes with C. pneumoniae activates the NF-κB binding to DNA (FIG. 7, column 2). Hydrocortisone and methylprednisolone, at 10⁻⁵ and 10⁻⁶ M, are without significant effect on the activation of NF-κB (columns 3-6).

NF-κB activation is considered as an important factor in the inflammatory reaction, leading to the expression of genes coding for inflammatory mediators (such as cytokines).

Activation of Gene Expression

By the PCR (<<polymerase chain reaction>>) method, it was observed that the incubation of monocytes with C. pneumoniae increased the expression of the genes (FIG. 8, column 2 compared to column 1) coding for interleukines 1β (IL-1β) (FIG. 8, part 1), 6 (IL-6) (FIG. 8, part 2) and 8 (IL-8) (FIG. 8, part 3), coding for inducible cyclo-oxygenase (COX-2) (FIG. 8, part 4) and coding for one of the subunits of NADPH-oxidase, the p22 protein (p₂₂^{phox}) (FIG. 8, part 5) (results are presented as relative values by comparison with a reference gene).

Searching Agents Able to Slow Down the Oxidant Activity of Chlamydia Infected Monocytes.

The enzymatic mechanism implicated in the oxidative metabolism of monocytes is thus mainly the NADPH-oxidase system: it was as such tried to moderate this enzyme activity in the conditions of the cell model, it is when the cells conditioned with Chlamydia increase their NADPH-oxidase activity.

On the previously described model, various drugs (well-known for their anti-inflammatory or their anti-oxidant action) were tested to verify if there is a possibility to moderate the production of reactive oxygen species by monocytes that are conditioned with Chlamydia; by acting either on NADPH-oxidase or its functioning mechanisms (such as the calcium mobilization), or by acting on the oxidant species themselves.

The drugs are added at the step of cell conditioning with Chlamydia. For comparison, some drugs were added to the Chlamydia-conditioned cells at the moment of the stimulation by PMA. The main drugs that were tested are:
The steroidal anti-inflammatory drugs: hydrocortisone, methylprednisolone, oestradiol.
the non steroidal anti-inflammatory drugs (NSAID): aspirin, indomethacin
the calcium metabolism modulators: acepromazine (phenothiazine), nifepidine (adalat)
antioxidants (oxydo-reduction stabilisators): tocopherol, apocynine
polyphenols: quercetin, rutin
coumarins: esculetin
statins
stilbenes.

Among all the pharmacological agents used, there is little or no effect of NSAIDs, acepromazine, adalat, vitamin E (tocopherol), apocyanine, statins and polyphenols: quercetin was active at quercetin was active at 10⁻⁴ M; this compound appears to be toxic at high concentrations, but toxicity does not exist at concentrations≦10⁻⁴ M) (Dunnick et al., Fundam Appl Toxicol 19: 423-431). The two groups of agents which are the most active in this cell model are glucocorticoids and stilbenes: the results are presented in % of the oxidation rate of control cells (cells conditioned with C. pneumoniae for 19 hours, and then stimulated with 10⁻⁷ M PMA). For the other tested pharmacological agents, the results are only presented by way of comparison with glucocorticoids and stilbenes.

Effects of Glucocorticoids

As the stimulation of monocytes is an early step in inflammation, it seems reasonable to study the effects of glucocorticoids, which are compounds well known for their anti-inflammatory and antioxidant activities. However, let us remember that glucocorticoids have been presented to facilitate the infection by Chlamydia, and that in the 1980's, glucocorticoids were suspected (without consistent argument) to favour atherosclerosis. Recently, the use of glucocorticoids has been proposed to slow down the atherosclerosis recurrences in arteries after stenting, balloon inflation angioplasty or vascular surgery (see pedagogic file on glucocorticoids). The glucocorticoids must be used at very low doses to moderate the cytokine production and the expression of adhesion molecules without developing immunosuppression in the patients.

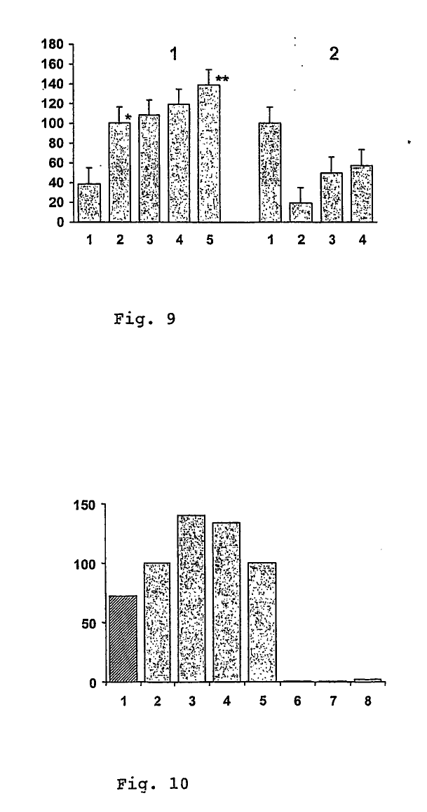

Two glucocorticoids were tested: hydrocortisone and methylprednisolone. Surprisingly, in said model, hydrocortisone (HCT) either does not modify the oxidant capacity, or even increases the oxidation rate (FIG. 9 part 1, columns 3, 4 and 5). On the contrary, HCT is an inhibitor on the oxidation rate when the cells are conditioned with endotoxins (LPS) of Escherichia coli (FIG. 9 part 2, columns 2, 3 and 4) (Ethylene values are expressed in % of the control cells=THP-1 cells stimulated by PMA 10⁻⁷ M after pre-incubation with Chlamydia (part 1, column 2) or LPS (part 1, column

*p<0.0001 versus column 1

**p≦0.05 versus column 2

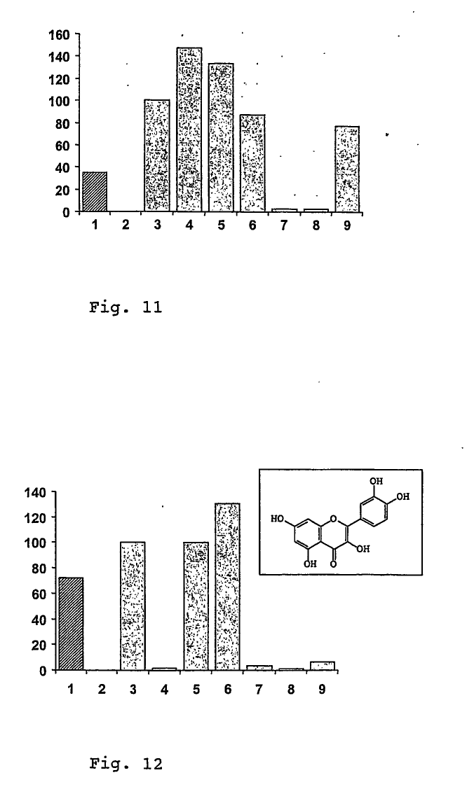

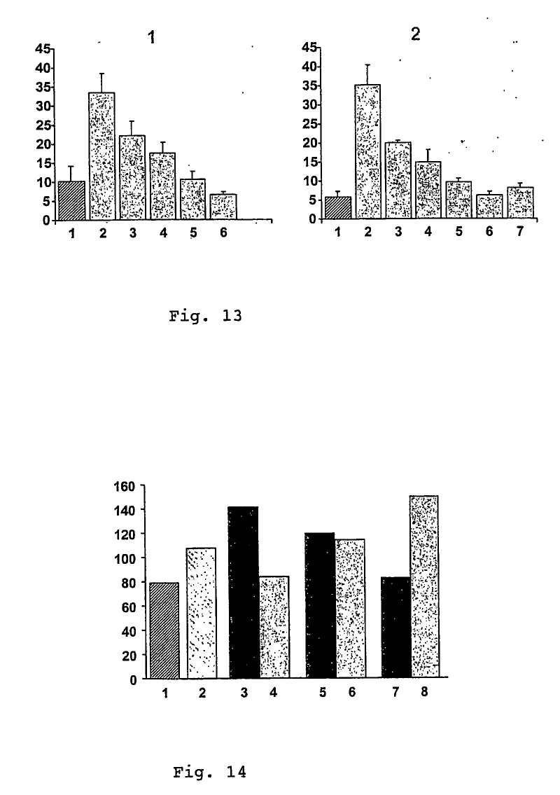

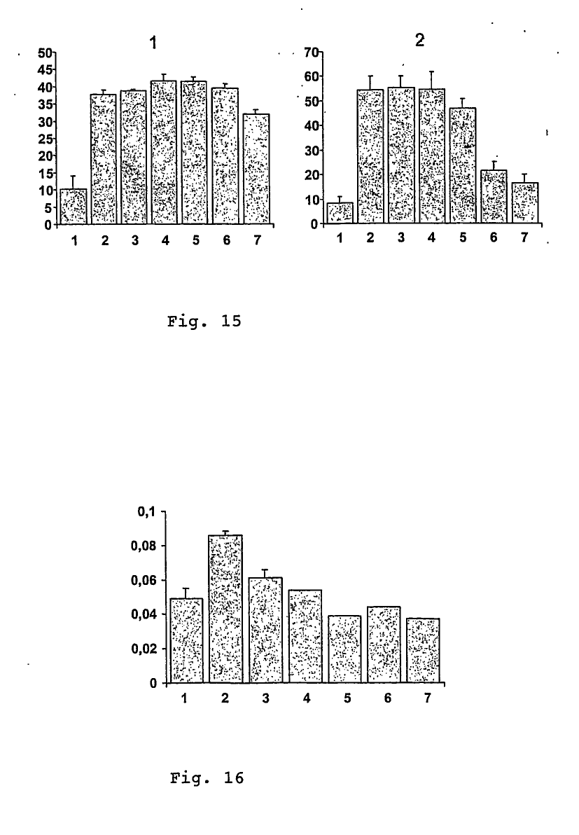

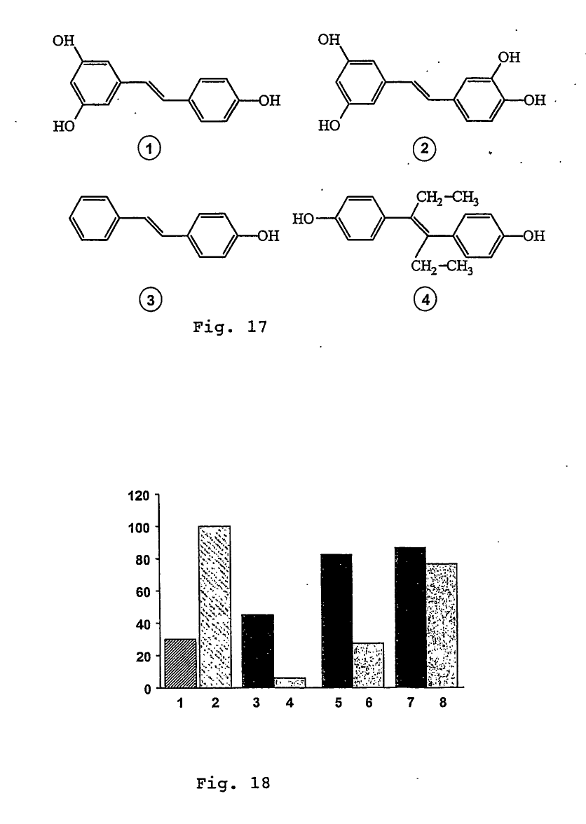

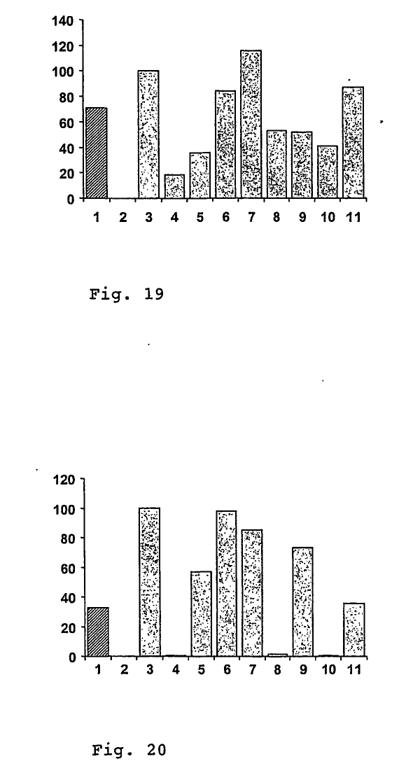

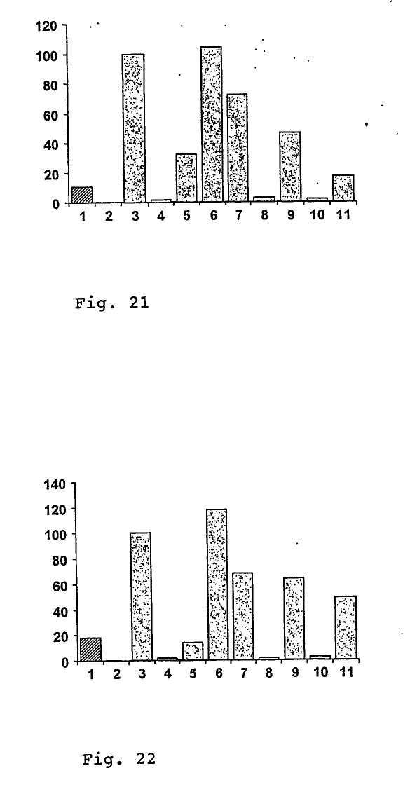

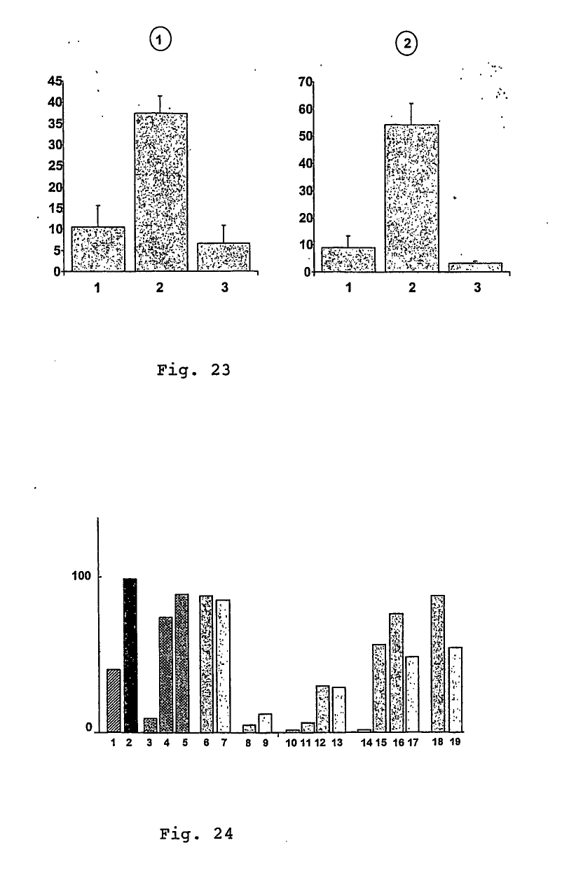

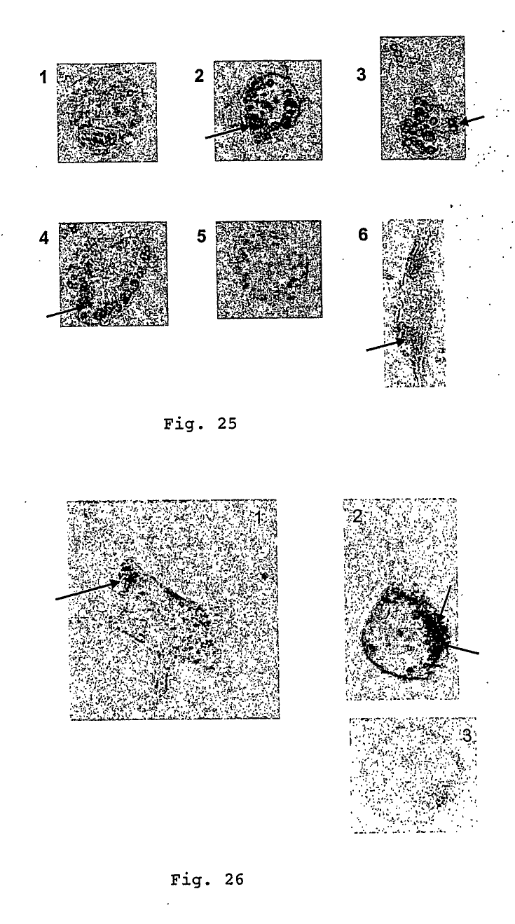

The effects of hydrocortisone were compared to those of oestradiol (FIG. 10), vitamin E (FIG. 11) and quercetin (FIG. 12), three pharmacological agents well-known for their antioxidant properties.

In FIG. 10, the values of ethylene are expressed in % of control (column 2) . In the columns 3, 4 and 5, the cells are pre-incubated with Chlamydia and oestradiol and in the columns 6, 7 and 8, oestradiol is added directly before the stimulation by PMA.

In FIG. 11, ethylene values are expressed in % of control (column 3) . In the columns 4, 5 and 6, the cells are pre-incubated with tocopherol and Chlamydia and in the columns 7, 8 and 9, tocopherol is added after pre-incubation with Chlamydia, directly before stimulation with PMA.

In FIG. 12, ethylene values are expressed in % of control (column 3) . In the columns 4, 5 and 6, quercetin is added at the time of pre-incubation with Chlamydia; and in the columns 7, 8 and 9, quercetin is added after the pre-incubation with Chlamydia, directly before stimulation by PMA.

When these drugs are pre-incubated with the monocytes (added at the same time as Chlamydia), oestradiol and tocopherol act in the same manner as HCT, favouring the oxidation rate (in a dose-dependent manner) (columns 3, 4 and 5 of FIG. 10, and columns 4, 5 and 6 of FIG. 11). Similar results were obtained with quercetin (FIG. 12, columns 5 and 6), except f or the 10⁻⁴ M concentration at which quercetin is an inhibitor (FIG. 12, column 4). However, this quercetin dose is near the toxic dose. On the contrary, when these three drugs are not pre-incubated with monocytes, but added directly before the stimulation by PMA, they quite completely inhibit the ethylene production, y a mechanism that can be attributed to their direct antioxidant effects (FIG. 10, columns 6, 7 and 8, and FIGS. 11 and 12, columns 7, 8 and 9).

In this model, hydrocortisone thus surprisingly acts by favouring the oxidant metabolism of monocytes conditioned with C. pneumoniae, contrary to the hydrocortisone effect on monocytes conditioned by LPS. This unexpected effect of hydrocortisone is also observed on the binding activity of NF-KB to DNA (FIG. 7, columns 3 and 5): at 10⁻⁶ M HCT, the activity of NF-κB even appears slightly stimulated in our cell model, an observation that is different from the data found in literature, which present HCT as inhibitor of NF-κB in LPS-conditioned cells.

On the production of the cytokines TNFα and IL-8, hydrocortisone has a dose-dependent inhibiting effect from 10⁻⁹ M to 10⁻⁵ M, but without a complete suppression of their production 1 (FIG. 13, parts 1 and 2).

Methylprednisolone exhibits effects similar to those of HCT: it either stimulates the oxidation processes or exerts no significant inhibition on the Chlamydia conditioned cells (FIG. 14). Ethylene values are expressed in % of control: THP-1 cells are pre-incubated with Chlamydia and then stimulated by PMA (FIG. 14, column 2). Methylprednisolone is either pre-incubated with the cells together with Chlamydia (FIG. 14, columns 3, 5 and 7) or added directly before the stimulation by PMA (FIG. 14, columns 4, 6 and 8).

However, methylprednisolone exerts less marked effects on the cytokine production: it does not inhibit TNFα production, except at 10⁻⁵ M (FIG. 15, part 1), and inhibits the IL-8 production at 10⁻⁵ and 10⁻⁶ M (FIG. 15, part 2). Methylprednisolone is also without significant effect on the activity of NF-κB (FIG. 7, columns 4 and 6).

The effects of HCT and methylprednisolone on the gene expression of p22^{phox}, one of the subunits NADPH-oxidase, was studied: the 2 glucocorticoids were found to be inhibitors (FIG. 16).

Effects of Stilbenes, Inhibitors of NADPH-Oxidase

Using resveratrol, a molecule of growing pharmacological importance, significant inhibitory effects were obtained on the production of oxidant species by Chlamydia-conditioned THP-1 after stimulation by PMA. Resveratrol is, by itself, an antioxidant, but, above all, it acts at the nuclear level and on the signal transduction. The resulting effect is a slowing down, dose-dependent, of the NADPH-oxidase activity.

In addition, the stilbenes shown in FIG. 17 were tested: they are all efficient, but only resveratrol and piceatanol are not carcinogenic; piceatanol has been less studied. Resveratrol appears as non toxic in chronic administration (in the rat) and convergent series of publications confirm its efficiency in the treatment of various human cancers. In vivo, resveratrol can be metabolized into piceatanol.

Resveratrol, preincubated together with Chlamydia, reduces the oxidizing activity of THP-1 cells (FIG. 18, columns 3, 5 and 7, and FIG. 19, columns 4 and 5). Its inhibiting capacity is still more marked when it is used immediately before the monocyte excitation by PMA, after the cells have been conditioned with Chlamydia pneumoniae (FIG. 18, columns 4, 6 and 8). Ethylene values are expressed in % of control: THP-1 cells are pre-incubated with Chlamydia and then stimulated by PMA (THP1+Chlamydia). Resveratrol is added either at pre-incubation together with Chlamydia (pre-incubation), or after pre-incubation directly before stimulation with PMA (no pre-incubation).

In this case, the inhibition is due to a direct antioxidant effect of resveratrol. The effects of the other stilbenes, used in pre-incubation together with Chlamydia, are shown in FIGS. 20, 21, and 22: they are all inhibitors. Ethylene values are expressed in % of control (column 3).

With microscopy, an inhibition of the monocyte transformation into macrophages by resveratrol is observed. This inhibition is nearly complete at 10⁻⁴ M and partial at 10⁻⁵ M.

Combined Effects of a Glucocorticoid and a Stilbene on the Moderation of the NADPH Oxidase Activity

Chlamydiae, living as parasites in host cells of the vascular wall, release toxins of which the nature is still undetermined but which, whatever they are, attract blood monocytes to the infected site. The attracted monocytes will not only react by a production of oxidant molecules, but also by the release of destroying proteases in the arterial tissue, and by the release of chemoattractive cytokines. These latter will continuously call other monocytes onto the infected site, which, in turn, will aggravate the destruction processes, and release new chemoattractive molecules: an amplification loop is then working. Stopping the release of cytokines and slowing down the monocytes/macrophages oxidizing activity is the only way to block this vicious circle.

With this purpose, the association of a corticosteroid (hydrocortisone), inhibitor of cytokine release, with a stilbene (resveratrol or piceatanol), which slows down the NADPH-oxidase activity, was tested. The associated compounds are used at 10⁻⁴ M or 10⁻⁵ M, and added to the cultures of THP-1 in pre-incubation with Chlamydia (FIGS. 19 and 20, columns 8, 9, 10, and 11). The effects of the association are compared to those exerted by each drug used alone (FIGS. 19 and 20, columns 4, 5, 6, and 7).

The best inhibitions of the oxidant production by Chlamydia-conditioned THP-1 and later stimulated by PMA, are obtained by the following associations: glucocorticoid 10⁻⁵ M/stilbene 10⁻⁴ M (FIGS. 19 and 20, column 10) and glucocorticoid 10⁻⁴ M/stilbene 10⁻⁴ M (FIGS. 19 and 20, column 8).

The associations glucocorticoid/bertrol and glucocorticoid/trans-4-stilbene are equally efficient (FIGS. 21 and 22, columns 8, 9, 10 and 11), but should not be retained until now in a therapeutic perspective, because these stilbenes are carcinogenic.

The combination glucocorticoid/stilbene is also efficient on the cytokines TNFα and IL-8 productions. FIG. 23 shows the results for TNFα (part 1) and for IL-8 (part 2 ) for the combination HCT 10⁻⁶ M/resveratrol 10⁻⁵ M.

Combined Effects of a Flavonoid and a Stilbene on the Moderation of the NADPH Oxidase Activity

In order to increase the efficacy of resveratrol, this compound (at doses ranging from 10⁻⁴ M to 10⁻⁷ M) was associated with a flavonoid, rutin at doses ranging from 10⁻⁴ M to 10⁻⁷ M or quercetin at doses ranging from 10⁻⁶ to 10⁻⁷ M. The associated compounds are added to the cultures of THP-1 in pre-incubation with Chlamydia (FIG. 24, columns 8 to 19). The effects of the association resveratrol/rutin or resveratrol/quercetin are compared to each drug used alone (resveratrol at 10⁻⁴ M, 10⁻⁵ M and 10⁻⁶ M: columns 3, 4 and 5 respectively; rutin at 10⁻⁶ M: column 6; quercetin at 10⁻⁶ M: column 7). The synergistic effect of the association is evident, and the association resveratrol/quercetin is still active at 10⁻⁷ M (column 19). The flavonoid acts by regeneration (reduction) of the oxidized resveratrol, it is by a redox phenomenon.

Development of a Model of Foam Cell Formation and Inhibition Tests by Resveratrol and Glucocorticoids

A key step in the development of the atherosclerotic plaque is the formation of foam cells, which are overloaded with lipids originating from the low density lipoproteins (LDL) that have been engulfed and oxidized in the cells.

A model of foam cell formation from monocytes (THP-1 cell line) was developed. The cells are incubated for 19 hours with Chlamydia pneumoniae. This incubation is followed by the addition of human LDL or liposomes, and another incubation period of 48 hours. At the end of this second period of incubation, the cells are fixed and stained with oil Red O (Sigma, Belgium) (Smirnova et al., 2004, Am J Physiol Heart Circ Physiol. March 11 (Epub ahead of print) to highlight the foam cells. When drugs are used in this model, they are added at the same time as the LDL or liposomes.

LDL are prepared from human blood drawn on EDTA. They are isolated by sequential flotation centrifugation with increased concentrations of KBr. After their isolation, LDL are dialysed, sterilised by filtration and their purity is confirmed by electrophoresis. Liposomes are lipid vesicles prepared by extrusion with cholesterol esters and phospholipids, mimicking lipoproteins.

Formation of the Foam Cells

The incubation of THP-1 with C. pneumoniae induces the differentiation of monocytes (FIG. 1.1) into macrophages (amiboid cells; 30 to 50 μm diameter), which are adherent to the surface of the culture flask (FIG. 1.2 and FIG. 25.1). By culturing the THP-1 monocytes with Chlamydia pneumoniae and LDL isolated from human plasma, a transformation of the monocytes into typical foam cells, characterised by numerous vacuoles filled with lipids stained by oil Red O (FIG. 25.2), was obtained.

Effects of Glucocorticoids

Hydrocortisone, added to the incubation medium, at 10⁻⁵ and 10⁻⁶ M, does not inhibit the transformation into foam cells. At 10⁻⁵ M, hydrocort:isone appears to have a favouring effect by increasing the number and size of the lipid vacuoles (FIG. 25.3 and 4).

Effects of Resveratrol

The addition of 10⁻⁵ M resveratrol to the incubation medium completely inhibits the transformation into foam cells (FIG. 25.5).

Effects of the Combination of Glucocorticoid and Resveratrol

On the model of monocyte transformation into foam cells, the resveratrol/hydrocortisone association was tested. Resveratrol 10⁻⁵ M+hydrocortisone 10⁻⁵ M considerably decreases the number of foam cells and improves their aspect (FIG. 25.6, to compare to 2 and 3).

Results were also obtained with an association of resveratrol and methylprednisolone: this association has effects that are similar to or even better than those obtained with the association of resveratrol with hydrocortisone.

Effects of the Combination of Resveratrol and Rutin

The monocytes, after incubation with Chlamydia pneumoniae, were transformed into characteristic macrophages (FIG. 26.1). The addition of liposomes further transformed the macrophages into foam cells with numerous Oil Red O stained lipid vacuoles (FIG. 26.2). When the incubation was performed with the association of resveratrol and rutin both at 10⁻⁶ M, the THP-1 cells did not transform into macrophages (the cells remained spherical and did not stick on the plates) and did not accumulate lipid vacuoles (FIG. 26.3).

1. A pharmaceutical composition comprising
an adequate pharmaceutical carrier;
a corticosteroid;
a stilbene-type phyto-alexin, a metabolite of said phyto-alexin, or a pharmaceutically acceptable salt of said phyto-alexin or its metabolite; and:
a polyphenol.

2. The composition of claim 1 wherein the phyto-alexin is selected from the group of resveratrol or a pharmaceutically acceptable salt thereof, piceatanol or a pharmaceutically acceptable salt thereof.

3. The composition according to claim 1 or 2, wherein the corticosteroid is methylprednisolone and/or hydrocortisone.

4. The composition according any of the claims 1 to 3, wherein the polyphenol is a flavonoid.

5. The composition according to the claim 4, wherein the flavonoid is a flavonoid according to formulae (1 and 2): 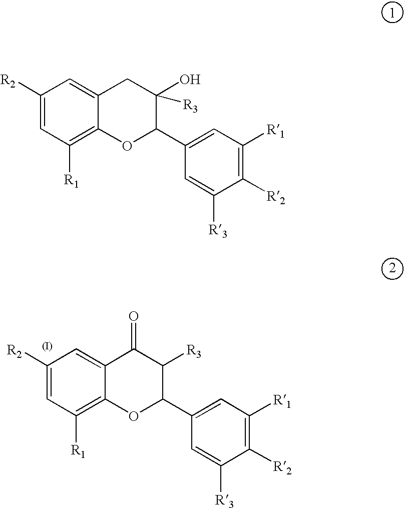
wherein R₁ can be H, OH; R₂ can be H, OH, O-sugar (preferably pentoses, hexoses saccharides); R₃ can be H, OH; R′₁ can be H, OH, OCH₃; R′₂ can be H, OH, OCH₃, OCH₂CH₂OH; and R′₃ can be H, OH, OCH₂CH₂OH.

6. The composition according to the claim 4 or 5, wherein the flavonoid is selected from the group consisting of quercetin, rutin, catechin, epicatechin, gallocatechin, leucocyanidin, hesperidin, kaempferin, myricetin, apigenin, diosmin, luteolin, fisetin, troxerutin, or a combination of two or more of the said flavonoids. With the composition according to the claims 6 comprising a flavonoid in a concentration range of about 10⁻⁵ M to about 10⁻⁶ M, preferably 10⁻⁶ M.

7. The composition according to the claim 2 comprising resveratrol and/or piceatanol in a concentration range of about 10⁻⁵ M to about 10⁻⁶ M, preferably 10⁻⁶ M.

8. The composition according to the claim 6, comprising flavonoids in a concentration range of about 10⁻⁵ M to about 10⁻⁶ M, preferably 10⁻⁶ M.

9. The composition according to the claim 3 comprising methylprednisolone in a concentration range of about 10⁻⁶ M×to about 10⁻⁸ M, preferably 10⁻⁷ M.

10. The composition according to the claim 9 comprising methylprednisolone in a concentration range of about 10⁻⁶ M×to about 10⁻⁵ M, preferably 10⁻⁷ M. As low as 10⁻⁶ M of 10⁻⁸ M in the presence of the stilbene-type phyto-alexin.

11. The pharmaceutical composition according to the claim 9, wherein the concentration of prednisolone or hydrocortisone can be as low as 10⁻⁷ M or 10⁻⁸ M in the presence of the stilbene-type phyto-alexin.

12. The pharmaceutical composition according to the claim 9 or 10, comprising prednisolone or hydrocortisone in a concentration range of about 10⁻⁶ to about 10⁻⁸ M and the stilbene-type phyto-alexin in a concentration range of about 10⁻⁵ M to about 10⁻⁶ M.

13. A patch, comprising a composition according to any of the preceding claims.

14. A formulation comprising the composition according to any of claims 1 to 11, which is in the form of a unit dosage.

15. A kit or a kit-in-parts comprising a composition or a formulation according to any of the preceding claims 1 to 13.

16. The kit or kit-in-parts according to claim 13, which further comprises an antibiotic, preferably a macrolide.

17. A treatment and/or prevention method of atherosclerosis from infectious origin, preferably human atherosclerosis from infectious origin, comprising the steps of administering to a subject in need thereof a composition or formulation according to any of the preceding claims.

18. The method according to claim 16, wherein the administration of composition or formulation according to any of the preceding claims is not combined with the administration of an antibiotic, not simultaneously and/or not separately.

19. The method according to claim 17, wherein the composition or formulation according to any of the preceding claims is administered over a period longer than one year.

20. The method according to claim 18, whereby the composition or formulation according to any of the preceding claims is administered over a period of years, but cut by periodic arrests of several weeks.

21. The method according to any of claims 16 to 19, wherein the composition or formulation according to any of the preceding claims is administered subcutaneously.

22. The method according to any of claims 16 to 20, combined with a separate antibiotic treatment, for instance a treatment with macrolides.

23. The method according to claim 21, wherein the antibiotic treatment is a continuous treatment, or is one with periodic arrests.

24. The method according to claim 22, wherein the composition or formulation according to any of the preceding claims is administered in a period of periodic antibiotic arrest.

25. The method according to any of the preceding claims wherein atherosclerosis is induced by endocellular micro-organisms such as Chlamydiae, Mycoplasmae, Bartonellae and/or CMV, preferably Chlamydiae.

26. Use of a composition or formulation according to any of the preceding claims 1 to 13 for the manufacture of a medicament to treat and/or prevent atherosclerosis from infectious origin, preferably human atherosclerosis from infectious origin induced by endocellular micro-organisms such as Chlamydiae, Mycoplasmae, Bartonellae and/or CMV.

27. A treatment and/or prevention method of atherosclerosis from infectious origin, preferably human atherosclerosis from infectious origin, comprising the steps of administrating to a subject in need thereof a composition comprising an adequate pharmaceutical carrier:
a corticosteroid;
a stilbene-type phyto-alexin, a metabolite of said phyto-alexin, or a pharmaceutically acceptable salt of said phyto-alexin or its metabolite.

28. The method of claim 26, wherein the phyto-alexin is selected from the group of resveratrol or a pharmaceutically acceptable salt thereof, piceatanol or a pharmaceutically acceptable salt thereof.

29. The method according to any of the preceding claims 25 to 30, comprising prednisolone or hydrocortisone in a concentration range of about 10⁻⁶ to about 10⁻⁸ M and the stilbene-type phyto-alexin in a concentration range of about 10⁻⁵ M to about 10⁻⁶ M.

30. Use of a composition of formulation comprising an adequate pharmaceutical carrier, a corticosteroid, a stilbene-type phyto-alexin, a metabolite of said phyto-alexin (or a pharmaceutically acceptable sort of said phyto-alexin or its metabolite). For the manufacture of a medicament for the treatment and/or the prevention of atherosclerosis from infectious origin, preferably human atherosclerosis from infectious origin induced by endocellular organisms such as Chlamydiae, Mycoplasmae, Bartonellae and/or CMV.

31. The use of claim 30, wherein the phyto-alexin is selected from the group consisting of resveratrol (or a pharmaceutically acceptable salt thereof), and piceatanol (or a pharmaceutically acceptable salt thereof.

32. The use of claim 30 or 31, wherein the corticosteroid is methylprednisolone and/or hydrocortisone comprising resveratrol and/or piceatanol in a concentration range of about 10⁻⁵ M to about 10⁻⁶ M, preferably 10⁻⁶ M.

33. The use according to the claim 32 comprising methylprednisolone in a concentration range of about 10⁻⁶ M to about 10⁻⁸ M, preferably these expose 10⁻⁷ M.

34. The use according to the claim 32 or 33, comprising resveratrol and/or piceatanol in a concentration range of about 10⁻⁵ M to about 10⁻⁶ M, preferably 10⁻⁶ M.

35. The use of claims 32 to 34, wherein the concentration of methylprednisolone or hydrocortisone can be as low as 10⁻⁸ M. These expose 10⁻⁷ M in the presence of the stilbene-type phyto-alexin.

36. The use according to any of the preceding claims 32 to 35, comprising prednisolone or hydrocortisone in a concentration range of about 10⁻⁷ M. These expose 10⁻⁶ M to about 10⁻⁸ M and a stilbene-type phyto-alexin in a concentration range of about 10⁻⁵ M to about 10⁻⁶ M.

37. The use according to any of the preceding claims 30 to 36, wherein the pharmaceutical composition is in the format of a patch.
